SEQUENCE LISTING (15 sequences)

SEQ ID NO: 1 - DNA, 24 bp, Artificial Sequence
  Description of Artificial Sequence Synthetic primer
tnaatccaggaatcctgctccatc

SEQ ID NO: 2 - DNA, 27 bp, Artificial Sequence
  Description of Artificial Sequence Synthetic primer
caaacagcccagttacaattacattag

SEQ ID NO: 3 - DNA, 24 bp, Artificial Sequence
  Description of Artificial Sequence Synthetic primer
cagataatgcatcgcttgctttag

SEQ ID NO: 4 - DNA, 25 bp, Artificial Sequence
  Description of Artificial Sequence Synthetic primer
tnggatgagcattcaacataccacg

SEQ ID NO: 5 - DNA, 23 bp, Artificial Sequence
  Description of Artificial Sequence Synthetic primer
ngataatgcatcgcttgctttag

SEQ ID NO: 6 - DNA, 20 bp, Artificial Sequence
  Description of Artificial Sequence Synthetic primer
gctgtttcctcatcaatccc

SEQ ID NO: 7 - DNA, 19 bp, Artificial Sequence
  Description of Artificial Sequence Synthetic primer
tngcgccgtaaagaaggtc

SEQ ID NO: 8 - DNA, 17 bp, Artificial Sequence
  Description of Artificial Sequence Synthetic primer
ctaccctgcgttgctca

SEQ ID NO: 9 - DNA, 21 bp, Artificial Sequence
  Description of Artificial Sequence Synthetic primer
tncatgtcgggggcatataac

SEQ ID NO: 10 - DNA, 19 bp, Artificial Sequence
  Description of Artificial Sequence Synthetic primer
tgcacctgaccatagaacg

SEQ ID NO: 11 - DNA, 25 bp, Artificial Sequence
  Description of Artificial Sequence Synthetic primer
ncctgctccatctgataatactcta

SEQ ID NO: 12 - DNA, 17 bp, Artificial Sequence
  Description of Artificial Sequence Synthetic primer
agcaggcaaggacagtg

SEQ ID NO: 13 - DNA, 18 bp, Artificial Sequence
  Description of Artificial Sequence Synthetic primer
tngcctgctgtgctgtgt

SEQ ID NO: 14 - DNA, 14 bp, Artificial Sequence
  Description of Artificial Sequence Synthetic primer
tcgtgcggtgcgtc

SEQ ID NO: 15 - DNA, 30 bp, Artificial Sequence
  Description of Artificial Sequence Synthetic primer
tcgtgcggtgcgtcacacagcacagcaggc

(19) United States
(12) Patent Application Publication
(10) Pub. No.: US 2012/0122095 A1
(43) Pub. Date: May 17, 2012

(54) Materials and methods for the detection of anthrax related toxin genes

(75) Inventors: Michael James Moser, Madison, WI (US); David J. Marshall, Madison, WI (US)

(73) Assignee: EraGen Biosciences, Inc.

(21) Appl. No.: 11/652,702

(22) Filed: Jan. 11, 2007

Related U.S. Application Data
(60) Provisional application No. 60/758,843, filed on Jan. 12, 2006.
(60) Provisional application No. 60/760,898, filed on Jan. 20, 2006.
(60) Provisional application No. 60/762,353, filed on Jan. 26, 2006.

(51) Int. Cl. C12Q 1/68; G01N 21/64

(52) U.S. Cl. 435/6.12; 435/6.15

(57) ABSTRACT

Disclosed are methods and kits for identifying a virulent bacteria in a sample, which may include virulent bacteria belonging to the Bacillus genus (e.g., Bacillus anthracis, Bacillus cereus, and Bacillus thuringiensis). Typically, the methods include (a) reacting a mixture that includes, in addition to nucleic acid isolated from the sample, (i) at least one oligonucleotide capable of specifically hybridizing to nucleic acid of plasmid pX01; and (ii) at least one oligonucleotide capable of specifically hybridizing to nucleic acid of plasmid pX02. In addition, the mixture may include control nucleic acid. In the methods, nucleic acid of plasmid pX01 and nucleic acid of plasmid pX02 are detected, and optionally control nucleic acid is detected, thereby identifying the virulent bacteria.

### CROSS-REFERENCE TO RELATED APPLICATIONS

This application claims the benefit of U.S. Provisional Application No. 60/758,843, filed Jan. 12, 2006, U.S. Provisional Application No. 60/760,898 filed on Jan. 20, 2006 and U.S. Provisional Application No. 60/762,353 filed on Jan. 26, 2006, all of which are incorporated by reference in their entirety.

### U.S. GOVERNMENT INTERESTS

This work was supported by Small Business innovation Research Grant No. AI052898 from the National Institute of Health and the National Institute of Allergy and Infectious Disease. U.S. Army Medical Research Institute of Infectious Diseases was supported by the U.S. Army Medical Research and Material Command (research plan 04-4-81-015). The Government has certain rights in this invention.

### BACKGROUND

The present methods relate generally to the field of identifying nucleic acids. In particular, the present methods relate to the field of identifying nucleic acids in a sample by detecting multiple signals such as signals emitted from fluorophores. The present methods also relate to the field of identifying nucleic acid in a sample by using labeled oligonucleotides to detect the nucleic acid in combination with agents for determining the melting temperature of the detected nucleic acid. The nucleic acids identified in the methods may be associated with virulence in bacteria.

Methods for detecting multiple nucleic acids such as multiplex methods are increasing important in medical diagnostics. Typical multiplex methods utilize PCR amplification, and in particular, real-time quantitative PCR. Real-time detection methods for PCR typically are based on one of two principles for monitoring amplification products: (1) specific hybridization by probes or primers to single-stranded DNA; or (2) binding by small molecules (e.g., intercalating agents) to double-stranded DNA. Probes and primers may include Molecular Beacon Probes, Scorpion® Primers, Taqman® Probes, LightCycler primers or probes and other labeled primers or probes. Small molecules that bind to DNA may include intercalators (e.g., SYBR™ Green I dye and ethidium bromide).

Methods for detecting nucleic acid that utilize probes and primers typically involve labeling each probe or primer with a unique label (e.g., a fluorescent dye). Multiplexing methods that utilize fluorescent dyes are often called “color multiplexing” methods. These methods require an instrument for detecting fluorescence from the multiple fluorophores, and the Roche LightCycler-1 is a commonly used clinical real-time PCR instrument.

Methods for detecting multiple nucleic acid based on melting temperature (“Tₘ”) typically utilize small binders such as intercalators. These methods often are called “Tₘ multiplexing” methods. Melting temperature analysis may include determining the melting temperature of a complex formed by a probe and the amplified target nucleic acid, or determining the melting temperature of the amplified target nucleic acid itself (i.e., determining the Tₘ of the amplicon). Intercalators for Tₘ, analysis typically exhibit a change in fluorescence based on whether the detected nucleic acid is double-stranded or single-stranded. Because intercalating agents interact with double-stranded nucleic acids non-specifically, multiple detected products must be distinguished by criteria such as resolvable melting temperatures.

Methods for detecting multiple nucleic acids are useful in the field of diagnostics. For example, methods for detecting multiple targets simultaneously may be useful for the detection of virulent bacteria (e.g., Bacillus that can cause anthrax), because it has been suggested that two plasmids confer virulence to a transformed bacteria. When genes of these two plasmids are present in bacteria other than Bacillus anthracis, the transformed bacteria may cause severe respiratory illness.

Accordingly, there is a need in the art for a rapid and sensitive method for the detection of such nucleic acids.

### SUMMARY

Disclosed are methods for identifying multiple nucleic acids in a sample. Typically, the methods include amplifying multiple nucleic acids and detecting multiple signals, such as signals emitted from fluorophores. In the disclosed methods, labeled oligonucleotides may be used to amplify multiple nucleic acids in the sample, for example as primers. In some embodiments, the methods include incorporating a label, such as a fluorophore or quencher, during amplification. The labels may be detected during amplification and/or during a melting step. For example, signals from the labels may be used to identify the multiple nucleic acids in the sample based on the melting temperatures of the multiple nucleic acids amplified during amplification (i.e., the multiple amplicons).

The multiple nucleic acids detected in the methods may include nucleic acids associated with bacterial virulence. For example, the multiple nucleic acids may include nucleic acids associated with plasmid pX01 and pX02. The target nucleic acid detected in the methods (e.g., pX01 and pX02) may be present on a plasmid in the bacteria or present within the genome of the bacteria. The multiple nucleic acids may include nucleic acid of cya (edema factor), lef (lethal factor), pagA (protective antigen), atxA and pagR. In some embodiments, the multiple nucleic acids may include nucleic acid that encodes the polypeptide encoded by the cya gene, the polypeptide encoded by the lef gene, the polypeptide encoded by the pagA gene, the polypeptide encoded by the atxA gene, and the polypeptide encoded by the pagR gene. The multiple nucleic acids may include nucleic acid of capB, capC, capA, dep, and acpA. In some embodiments, the multiple nucleic acids may include nucleic acid that encodes the polypeptide of the capB gene, the polypeptide of the capC gene, the polypeptide of the capA gene, the polypeptide of the dep gene, and the polypeptide of the acpA gene.

As disclosed herein, methods of detecting virulent bacteria in a sample, wherein the virulent bacteria include pX01 and pX02 nucleic acid, may include: a) amplifying the pX01 and pX02 nucleic acid, if present in the sample, with first and second primer pairs to provide amplification products, wherein at least one primer of the first primer pair specifically hybridizes to pX01 nucleic acid, and at least one primer of the second primer pair specifically hybridizes to pX02 nucleic acid, and at least one primer of each primer pair includes a first non-natural base and a first label; b) incorporating a second non-natural base into the amplification products, wherein the second non-natural base base-pairs with the first non-natural base and the second non-natural base is coupled to a second label; c) observing a signal during amplification thereby detecting and quantifying the pX01 and pX02 nucleic acid in the sample.

In some embodiments, the virulent bacteria may be a member of the Bacillus genus, and may include, for example Bacillus anthracis.

In other embodiments, at least one primer of the first primer pair may be capable of specifically hybridizing to a nucleic acid sequence which may include one or more of the following: cya nucleic acid sequence, lef nucleic acid sequence, pagA nucleic acid sequence, atxA nucleic acid sequence, and pagR nucleic acid sequence. In some embodiments, cya nucleic acid sequence and pagA nucleic acid sequence may be preferred. In still other embodiments, at least one primer from the second primer pair may be capable of specifically hybridizing to a nucleic acid sequence which may include one or more of the following: capB nucleic acid sequence, capC, nucleic acid sequence, capA nucleic acid sequence, dep nucleic acid sequence, and acpA nucleic acid sequence. In some embodiments, capB nucleic acid sequence may be preferred.

In some embodiments, the first non-natural base may be iso-C or iso-G. In other embodiments, the second non-natural base may be the other of iso-C or iso-G.

In still other embodiments, the first label may include a fluorophore and the second label may include a quencher. In further embodiments, at least one primer of each primer pair may include a different fluorophore.

In some methods, an internal control may be included. For example, in some embodiments, the method may include (d) amplifying an internal control nucleic acid to provide a control amplification product, and (e) detecting the internal control nucleic acid.

Another method of detecting a virulent bacteria in a sample, wherein the virulent bacteria include pX01 and pX02 nucleic acid, may include: a) reacting a mixture that includes: (i) the sample; (ii) a first oligonucleotide primer which may include a sequence complementary to the pX01 nucleic acid, a first non-natural base, and a first label; (iii) a second oligonucleotide primer which may include a sequence complementary to the pX02 nucleic acid, a second non-natural base, and a second label; and (iv) a nucleotide comprising a third non-natural base and a quencher, wherein the third non-natural base base-pairs with the first and second non-natural bases; b) amplifying the pX01 and pX02 nucleic acid, if present in the sample, to generate labeled amplification products; and c) observing a signal from the first label, the second label, or both labels during amplification thereby detecting the virulent bacteria in the sample.

In some embodiments, the virulent bacteria may be a member of the Bacillus genus, and may include, for example Bacillus anthracis.

In some embodiments, the first oligonucleotide primer may specifically hybridize to a nucleic acid sequence including one or more of the following: cya nucleic acid sequence, lef nucleic acid sequence, pagA nucleic acid sequence, atxA nucleic acid sequence, and pagR nucleic acid sequence. In other embodiments, the second primer may specifically hybridizes to a nucleic acid sequence including one or more of the following: capB nucleic acid sequence, capC, nucleic acid sequence, capA nucleic acid sequence, dep nucleic acid sequence and acpA nucleic acid sequence. In still other embodiments, the first oligonucleotide primer may specifically hybridizes to a nucleic acid sequence including cya nucleic acid sequence or pagA nucleic acid sequence, and the second oligonucleotide primer may specifically hybridizes to a capB nucleic acid sequence.

In still further embodiments, the first non-natural base and the second non-natural base may be iso-C or iso-G, and the third non-natural base may be the other of iso-C or iso-G. In other embodiments, the first label may include a fluorophore and the second label include a different fluorophore.

In some methods, an internal control may be included. For example, in some embodiments, the method may include (d) amplifying an internal control nucleic acid to provide a control amplification product, and (e) detecting the internal control nucleic acid.

In further embodiments, kits are provided for the detection of virulent bacteria. For example such a kit may include: a) a first oligonucleotide primer comprising a sequence complementary to the pX01 nucleic acid, a first non-natural base, and a first label (e.g., a first fluorophore); b) a second oligonucleotide primer comprising a sequence complementary to the pX02 nucleic acid, a second non-natural base, and a second label (e.g., a second fluorophore); and c) a nucleotide comprising a third non-natural base and a third label (e.g., a quencher for first and second fluorophores of (a) and (b)), wherein the third non-natural base base-pairs with the first and second non-natural bases.

As disclosed herein, other methods for identifying a virulent bacteria in a sample may include: (a) reacting a mixture that includes (i) nucleic acid isolated from the sample, (ii) at least one oligonucleotide capable of specifically hybridizing to nucleic acid of plasmid pX01, and (iii) at least one oligonucleotide capable of specifically hybridizing to nucleic acid of plasmid pX02; (b) detecting nucleic acid of plasmid pX01; and (c) detecting nucleic acid of plasmid pX02. The virulent bacteria may be a member of the Bacillus genus (e.g., Bacillus anthracis, Bacillus cereus, and Bacillus thuringiensis). In some embodiments of the methods, the reaction mixture further may include (iv) internal control nucleic acid; and (v) at least one oligonucleotide capable of specifically hybridizing to the internal control nucleic acid. In these embodiments, the methods further may include (d) detecting the internal control nucleic acid nucleic acid.

In the methods, the at least one oligonucleotide that is capable of specifically hybridizing to nucleic acid of plasmid pX01 may be capable of specifically hybridizing to at least one of nucleic acid of cya (edema factor), nucleic acid of lef (lethal factor), nucleic acid of pagA (protective antigen), nucleic acid of atxA, and nucleic acid of pagR. Desirably, the at least one oligonucleotide is capable of specifically hybridizing to nucleic acid of cya (edema factor) or pagA (protective antigen).

In the methods, the at least one oligonucleotide that is capable of specifically hybridizing to nucleic acid of plasmid pX02 may be capable of specifically hybridizing to at least one of nucleic acid of capB, nucleic acid of capC, nucleic acid of capA, nucleic acid of dep, and nucleic acid of acpA. Desirably, the at least one oligonucleotide is capable of specifically hybridizing to nucleic acid of capB.

The methods may include amplifying at least one of nucleic acid of plasmid pX01, nucleic acid of plasmid pX02, and control nucleic acid. For example, the reaction mixture may include at least two oligonucleotides capable of specifically hybridizing to nucleic acid of plasmid pX01, nucleic acid of pX02, or control nucleic acid where the two oligonucleotides are capable of functioning as primers. In some embodiments, the reaction mixture includes three pairs of oligonucleotides capable of specifically hybridizing to the nucleic acid of plasmid pX01, nucleic acid of pX02, and control nucleic acid, respectively, where the three pairs of primers are capable of functioning as primers.

In the methods, where a pair of oligonucleotides is used to amplify a target nucleic acid (e.g., nucleic acid of pX01, nucleic acid of pX02, or control nucleic acid), at least one of the pair of oligonucleotides may include a label. In some embodiments, at least one of the pair of oligonucleotides may include at least one nucleotide other than A, C, G, T, and U, or a non-natural nucleotide. Non-natural nucleotides are described in U.S. patent application publication 2002-0150900, which is incorporated herein by reference in its entirety. Non-natural nucleotides may include iso-cytosine and iso-guanine (i.e., “iC” and “iG,” respectively). In further embodiments, the label may include a fluorophore and the amplification mixture may include at least one nucleotide covalently linked to a quencher (e.g., Dabcyl where the fluorophore may include a fluorophore capable of being quenched by Dabcyl). The nucleotide covalently linked to the quencher may include non-natural nucleotides (e.g., iC and iG).

In some embodiments, the methods for detecting a virulent bacteria in a sample may include: (a) reacting a mixture that includes, (i) nucleic acid isolated from the sample, (ii) a first pair of oligonucleotides capable of specifically hybridizing to nucleic acid of plasmid pX01, where at least one oligonucleotide of the first pair includes a first label, (iii) a second pair of oligonucleotides capable of specifically hybridizing to nucleic acid of plasmid pX02, where at least one oligonucleotide of the second pair includes a second label, (iv) control nucleic acid, and (v) a third pair of oligonucleotides capable of specifically hybridizing to the control nucleic acid, where at least one oligonucleotide of the third pair includes a third label. In desirable embodiments, the first label, second label, and third label are different. The method typically further includes: (b) amplifying and detecting (i) the nucleic acid of plasmid pX01, (ii) nucleic acid of plasmid pX02, and (iii) the control nucleic acid. In some embodiments, the first label, second label, and third label include three different fluorophores and the reaction mixture further includes an amplification mixture. The amplification mixture may include a nucleotide covalently linked to a quencher capable of quenching the three different fluorophores.

The methods described herein further may include determining a melting temperature for an amplicon (e.g., amplified nucleic acid of at least one of amplified nucleic acid of plasmid pX01, amplified nucleic acid of plasmid pX02, and amplified control nucleic acid). The melting temperature may be determining by exposing the amplicon to a gradient of temperatures and observing a signal from a reporter. Optionally, the melting temperature may be determined by (a) reacting an amplicon with an intercalating agent at a gradient of temperatures and (b) observing a detectable signal from the intercalating agent.

The methods may be performed in any suitable reaction chamber under any suitable conditions. For example, the methods may be performed in a reaction chamber without opening the reaction chamber. The reaction chamber may be part of an array or reaction chambers. In some embodiments, the steps of the methods may be performed separately in different reaction chambers.

Other kits for performing the methods disclosed herein may include at least one component for performing the methods.

For example, kits may include (a) a first pair of oligonucleotides capable of specifically hybridizing to nucleic acid of plasmid pX01, where at least one oligonucleotide of the first pair includes a first label; and (b) a second pair of oligonucleotides capable of specifically hybridizing to nucleic acid of plasmid pX02, where at least one oligonucleotide of the second pair includes a second label. Desirably, the first label and second label are different. Kits further may include (c) control nucleic acid; and (d) a third pair of oligonucleotides capable of specifically hybridizing to the control nucleic acid, where at least one oligonucleotide of the third pair includes a third label. Desirably, the first label, second label, and third label are different.

In some embodiments of the kits, the first pair of oligonucleotides may be capable of specifically hybridizing to nucleic acid selected from nucleic acid of cya (edema factor), nucleic acid of lef (lethal factor), nucleic acid of pagA (protective antigen), nucleic acid of atxA, and nucleic acid of pagR. Desirably, the first pair of oligonucleotides is capable of specifically hybridizing to nucleic acid selected from nucleic acid of cya (edema factor) or nucleic acid of pagA (protective antigen).

In some embodiments of the kits, the second pair of oligonucleotides may capable of specifically hybridizing to nucleic acid selected from nucleic acid of capB, nucleic acid of capC, nucleic acid of capA, nucleic acid of dep, and nucleic acid of acpA. Desirably, the second pair of oligonucleotides is capable of specifically hybridizing to nucleic acid of capB.

In further embodiments of the kits, at least one oligonucleotide of the first, second, and third pair of oligonucleotides may include at least one nucleotide other than A, C, G, T, and U (e.g., iC and iG). In still further embodiments of the kits, the first label, second label, and third label may include three different fluorophores and the kit may further include an amplification mixture. Desirably, the amplification mixture includes a nucleotide covalently linked to a quencher capable of quenching the three different fluorophores. The nucleotide covalently linked to a quencher may include nucleotide other than A, C, G, T, and U (e.g., iC and iG). The amplification mixture may include an enzyme desirable for performing PCR (e.g., Taq polymerase).

In some embodiments of the kits, the kits further include a reagent for determining a melting temperature of nucleic acid. The reagent may include an intercalating agent such as SYBR dyes.

### BRIEF DESCRIPTION OF THE DRAWINGS

FIG. 1 MultiCode RTx system schematic. Targets are amplified with a standard reverse primer and a forward primer that contains a single iC nucleotide and a fluorescent reporter. Amplification is performed in the presence of dabcyl-diGTP. Site-specific incorporation places the quencher in close proximity to the reporter that leads to a decrease in fluorescence.

FIG. 2 Linear Curve Analysis. The two RTx systems pagA:capB:IPC (A and B) and cya:capB:IPC (C and D) were tested for linearity for both corresponding synthetic targets using ten-fold dilution series from 3 to 3×10⁵ copies in duplicate on different days. Top panels show linear curve analyses of log copy number vs cycle threshold (Ct). Bottom panels show real-time RTx data in relative fluorescence units (RFU) vs. PCR cycles. Internal positive control is not shown.

FIG. 3 MultiCode RTx data from Limit of Detection Study. Ten-fold dilution series from 1 pg to 1 fg of B. anthracis Ames total genomic DNA was used in duplicate. Data is provided for the cya:capB:IPC multiplex assay. Limit of detection for cya primer set was 10 fg or ˜2 copies.

### DETAILED DESCRIPTION

Disclosed are methods and kits for detecting multiple nucleic acids in a sample. Typically, the methods include detecting multiple signals such as a signal emitted from a fluorophore. Also disclosed herein are oligonucleotides, especially primers and probes, which may be used for the detection of anthrax toxin-encoding sequences. The methods, kits, and oligonucleotides disclosed herein may be used to detect pathogenic bacilli (e.g., B. anthracis) containing genes whose products are toxic to humans.

As used herein, unless otherwise stated, the singular forms “a,” “an,” and “the” include plural reference. Thus, for example, a reference to “an oligonucleotide” includes a plurality of oligonucleotide molecules, and a reference to “a nucleic acid” is a reference to one or more nucleic acids.

As used herein, the term “sample” is used in its broadest sense. A sample may include a bodily tissue or a bodily fluid including but not limited to blood (or a fraction of blood such as plasma or serum), lymph, mucus, tears, urine, and saliva. A sample may include an extract from a cell, a chromosome, organelle, or a virus. A sample may comprise DNA (e.g., genomic DNA), RNA (e.g., mRNA), and cDNA, any of which may be amplified to provide amplified nucleic acid. A sample may include nucleic acid in solution or bound to a substrate (e.g., as part of a microarray). A sample may comprise material obtained from an environmental locus (e.g., a body of water, soil, and the like) or material obtained from a fomite (i.e., an inanimate object that serves to transfer pathogens from one host to another).

As used herein, the term “microarray” refers to an arrangement of a plurality of polynucleotides, polypeptides, or other chemical compounds on a substrate. The terms “element” and “array element” refer to a polynucleotide, polypeptide, or other chemical compound having a unique and defined position on a microarray.

As used herein, an oligonucleotide is understood to be a molecule that has a sequence of bases on a backbone comprised mainly of identical monomer units at defined intervals. The bases are arranged on the backbone in such a way that they can enter into a bond with a nucleic acid having a sequence of bases that are complementary to the bases of the oligonucleotide. The most common oligonucleotides have a backbone of sugar phosphate units. A distinction may be made between oligodeoxyribonucleotides (“dNTP's”), which do not have a hydroxyl group at the 2′ position, and oligoribonucleotides (“NTP's”), which have a hydroxyl group in this position. Oligonucleotides also may include derivatives, in which the hydrogen of the hydroxyl group is replaced with organic groups, e.g., an allyl group.

In some embodiments, oligonucleotides as described herein may include a peptide backbone. For example, the oligonucleotides may include peptide nucleic acids or “PNA.” Peptide nucleic acids are described in WO 92/20702, which is incorporated herein by reference.

An oligonucleotide is a nucleic acid that includes at least two nucleotides. Oligonucleotides used in the methods disclosed herein typically include at least about ten (10) nucleotides and more typically at least about fifteen (15) nucleotides. In some embodiments, oligonucleotides for the methods disclosed herein include about 10-25 nucleotides. An oligonucleotide may be designed to function as a “primer.” A “primer” is a short nucleic acid, usually a ssDNA oligonucleotide, which may be annealed to a target polynucleotide by complementary base-pairing. The primer may then be extended along the target DNA strand by a DNA polymerase enzyme. Primer pairs can be used for amplification (and identification) of a nucleic acid sequence (e.g., by the polymerase chain reaction (PCR)). An oligonucleotide may be designed to function as a “probe.” A “probe” refers to an oligonucleotide, its complements, or fragments thereof, which is used to detect identical, allelic or related nucleic acid sequences. Probes may include oligonucleotides which have been attached to a detectable label or reporter molecule. Typical labels include fluorescent dyes, radioactive isotopes, ligands, chemiluminescent agents, and enzymes.

An oligonucleotide may be designed to be specific for a target nucleic acid sequence in a sample. For example, an oligonucleotide may be designed to include “antisense” nucleic acid sequence of the target nucleic acid. As used herein, the term “antisense” refers to any composition capable of base-pairing with the “sense” (coding) strand of a specific target nucleic acid sequence. An antisense nucleic acid sequence may be “complementary” to a target nucleic acid sequence. As used herein, “complementarity” describes the relationship between two single-stranded nucleic acid sequences that anneal by base-pairing. For example, 5′-AGT-3′ pairs with its complement, 3′-TCA-5′.

Oligonucleotides as described herein typically are capable of forming hydrogen bonds with oligonucleotides having a complementary base sequence. These bases may include the natural bases such as A, G, C, T and U, as well as artificial bases such as deaza-G. As described herein, a first sequence of an oligonucleotide is described as being 100% complementary with a second sequence of an oligonucleotide when the consecutive bases of the first sequence (read 5′->3′) follow the Watson-Crick rule of base pairing as compared to the consecutive bases of the second sequence (read 3′->5′). An oligonucleotide may include nucleotide substitutions. For example, an artificial base may be used in place of a natural base such that the artificial base exhibits a specific interaction that is similar to the natural base.

An oligonucleotide that is specific for a target nucleic acid also may be specific for a nucleic acid sequence that has “homology” to the target nucleic acid sequence. As used herein, “homology” refers to sequence similarity or, interchangeably, sequence identity, between two or more polynucleotide sequences or two or more polypeptide sequences. The terms “percent identity” and “% identity” as applied to polynucleotide sequences, refer to the percentage of residue matches between at least two polynucleotide sequences aligned using a standardized algorithm (e.g., BLAST).

An oligonucleotide that is specific for a target nucleic acid will “hybridize” to the target nucleic acid under suitable conditions. As used herein, “hybridization” or “hybridizing” refers to the process by which a oligonucleotide single strand anneals with a complementary strand through base pairing under defined hybridization conditions. “Specific hybridization” is an indication that two nucleic acid sequences share a high degree of complementarity. Specific hybridization complexes form under permissive annealing conditions and remain hybridized after any subsequent washing steps. Permissive conditions for annealing of nucleic acid sequences are routinely determinable by one of ordinary skill in the art and may occur, for example, at 65° C. in the presence of about 6×SSC. Stringency of hybridization may be expressed, in part, with reference to the temperature under which the wash steps are carried out. Such temperatures are typically selected to be about 5° C. to 20° C. lower than the thermal melting point (Tₘ) for the specific sequence at a defined ionic strength and pH. The Tₘ, is the temperature (under defined ionic strength and pH) at which 50% of the target sequence hybridizes to a perfectly matched probe. Equations for calculating Tₘ, and conditions for nucleic acid hybridization are known in the art.

As used herein, “nucleic acid,” “nucleotide sequence,” or “nucleic acid sequence” refer to a nucleotide, oligonucleotide, polynucleotide, or any fragment thereof and to naturally occurring or synthetic molecules. These phrases also refer to DNA or RNA of genomic or synthetic origin which may be single-stranded or double-stranded and may represent the sense or the antisense strand, or to any DNA-like or RNA-like material. An “RNA equivalent,” in reference to a DNA sequence, is composed of the same linear sequence of nucleotides as the reference DNA sequence with the exception that all occurrences of the nitrogenous base thymine are replaced with uracil, and the sugar backbone is composed of ribose instead of deoxyribose. RNA may be used in the methods described herein and/or may be converted to cDNA by reverse-transcription for use in the methods described herein.

As used herein, “amplification” or “amplifying” refers to the production of additional copies of a nucleic acid sequence. Amplification is generally carried out using polymerase chain reaction (PCR) technologies known in the art. The term “amplification reaction system” refers to any in vitro means for multiplying the copies of a target sequence of nucleic acid. The term “amplification reaction mixture” refers to an aqueous solution comprising the various reagents used to amplify a target nucleic acid. These may include enzymes (e.g., a thermostable polymerase), aqueous buffers, salts, amplification primers, target nucleic acid, and nucleoside triphosphates, and optionally at least one labeled probe and/or optionally at least one agent for determining the melting temperature of an amplified target nucleic acid (e.g., a fluorescent intercalating agent that exhibits a change in fluorescence in the presence of double-stranded nucleic acid).

During PCR, the polymerase enzyme, first primer and second primer are used to generate an amplification product as described herein. One PCR technique that can be used is a modified PCR, or Fast-shot™ amplification. As used herein, the term “Fast-shot™ amplification” refers to a modified polymerase chain reaction.

Traditional PCR methods include the following steps: denaturation, or melting of double-stranded nucleic acids; annealing of primers; and extension of the primers using a polymerase. This cycle is repeated by denaturing the extended primers and starting again. The number of copies of the target sequence in principle grows exponentially. In practice, it typically doubles with each cycle until reaching a plateau at which more primer-template accumulates than the enzyme can extend during the cycle; then the increase in target nucleic acid becomes linear.

Fast-shot amplification is a modified polymerase chain reaction wherein the extension step, as well as the annealing and melting steps, are very short or eliminated. As used herein, when referring to “steps” of PCR, a step is a period of time during which the reaction is maintained at a desired temperature without substantial fluctuation of that temperature. For example, the extension step for a typical PCR is about 30 seconds to about 60 seconds. The extension step for a Fast-shot™ amplification typically ranges from about 0 seconds to about 20 seconds. Preferably, the extension step is about 1 second or less. In a preferred embodiment, the extension step is eliminated. The time for annealing and melting steps for a typical PCR can range from 30 seconds to 60 seconds. The time for annealing and melting steps for a Fast-shot™ amplification generally can range from about 0 seconds to about 60 seconds. For Fast-shot™ amplification, the annealing and melting steps are typically no more than about 2 seconds, preferably about 1 second or less. When the extension step is eliminated, the temperature is cycled between the annealing and melting steps without including an intermediate extension step between the annealing and melting temperatures.

Additionally, the limit of how quickly the temperature can be changed from the annealing temperature to the melting temperature depends upon the efficiency of the polymerase in incorporating bases onto an extending primer and the number of bases it must incorporate, which is determined by the gap between the primers and the length of the primers. Examples of Fast-shot™ amplification are shown in the Examples.

The number of Fast-shot™ amplification cycles required to determine the presence of a nucleic acid sequence in a sample can vary depending on the number of target molecules in the sample. In one of the examples described below, a total of 37 cycles was adequate to detect as little as 100 target nucleic acid molecules.

The amplification methods described herein may include “real-time monitoring” or “continuous monitoring.” These terms refer to monitoring multiple times during a cycle of PCR, preferably during temperature transitions, and more preferably obtaining at least one data point in each temperature transition. The term “homogeneous detection assay” is used to describe an assay that includes coupled amplification and detection, which may include “real-time monitoring” or “continuous monitoring.”

Amplification of nucleic acids may include amplification of nucleic acids or subregions of these nucleic acids. For example, amplification may include amplifying portions of nucleic acids between 100 and 300 bases long by selecting the proper primer sequences and using the PCR.

PCR may be used to generate an amplification product (i.e., an amplicon). Some amplicons may comprise a double-stranded region and a single-stranded region. The double-stranded region may result from extension of the first and second primers. The single-stranded region may result from incorporation of a non-natural base in the second primer of the disclosed methods. A region of the first and/or second primer may not be complementary to the target nucleic acid. Because the non-natural base follows base-pairing rules of Watson and Crick and forms bonds with other non-natural bases, the presence of a non-natural base may maintain a region as a single-stranded region in the amplification product.

In an alternative embodiment, the single-stranded region may comprise more than one non-natural base. The number of non-natural bases included in the first and/or second amplification primer can be selected as desired.

The disclosed methods may include amplifying at least one nucleic acid in the sample, at least two nucleic acids, or at least three nucleic acids. In the disclosed methods, amplification may be monitored using real-time methods. Amplification mixtures may include natural nucleotides (e.g., A, C, G, T, and U) and non-natural nucleotides (e.g., iC and iG). Non-natural nucleotides and bases are described in U.S. patent application publication 2002-0150900 and U.S. Pat. No. 6,997,161 both of which are incorporated herein by reference in their entirety. The nucleotides, which may include non-natural nucleotides, may include a label (e.g., a quencher or a fluorophore).

As noted previously, the oligonucleotides of the present methods may function as primers. In some embodiments, the oligonucleotides are labeled. For example, the oligonucleotides may be labeled with a reporter that emits a detectable signal (e.g., a fluorophore). The oligonucleotides may include at least one non-natural nucleotide. For example, the oligonucleotides may include at least one nucleotide that is not A, C, G, T, or U (e.g., iC or iG). Where the oligonucleotide is used as a primer for PCT, the amplification mixture may include at least one nucleotide that is labeled with a quencher (e.g., Dabcyl). The labeled nucleotide may include at least one non-natural nucleotide. For example, the labeled nucleotide may include at least one nucleotide that is not A, C, G, T, or U (e.g., iC or iG).

In some embodiments, the oligonucleotide may be designed not to form an intramolecular structure such as a hairpin. In other embodiments, the oligonucleotide, may be designed to form an intramolecular structure such as a hairpin. For example, the oligonucleotide may be designed to form a hairpin structure that is altered after the oligonucleotide hybridizes to a target nucleic acid, and optionally, after the target nucleic acid is amplified using the oligonucleotide as a primer.

The oligonucleotide may be labeled with a fluorophore that exhibits quenching when incorporated in an amplified product as a primer. In other embodiments, the oligonucleotide may emit a detectable signal after the oligonucleotide is incorporated in an amplified product as a primer (e.g., inherently, or by fluorescence induction or fluorescence dequenching). Such primers are known in the art (e.g., LightCycler primers, Amplifluor® Primers, Scorpion® Primers and Lux™ Primers). The fluorophore used to label the oligonucleotide may emit a signal when intercalated in double-stranded nucleic acid. As such, the fluorophore may emit a signal after the oligonucleotide is used as a primer for amplifying the nucleic acid. In some embodiments, the fluorescent dye may function as a fluorescence donor for fluorescence resonance energy transfer (FRET). The detectable signal may be quenched when the oligonucleotide is used to amplify a target nucleic acid. For example, the amplification mixture may include nucleotides that are labeled with a quencher for the detectable signal emitted by the fluorophore. Optionally, the oligonucleotides may be labeled with a second fluorescent dye or a quencher dye that may function as a fluorescence acceptor (e.g., for FRET). Where the oligonucleotide is labeled with a first fluorescent dye and a second fluorescent dye, a signal may be detected from the first fluorescent dye, the second fluorescent dye, or both.

The disclosed methods may be performed with any suitable number of oligonucleotides. Where a plurality of oligonucleotides are used (e.g., two or more oligonucleotides), different oligonucleotide may be labeled with different fluorescent dyes capable of producing a detectable signal. In some embodiments, oligonucleotides are labeled with at least one of two different fluorescent dyes. In further embodiments, oligonucleotides are labeled with at least one of three different fluorescent dyes.

In some embodiments, each different fluorescent dye emits a signal that can be distinguished from a signal emitted by any other of the different fluorescent dyes that are used to label the oligonucleotides. For example, the different fluorescent dyes may have wavelength emission maximums all of which differ from each other by at least about 5 nm (preferably by least about 10 nm). In some embodiments, each different fluorescent dye is excited by different wavelength energies. For example, the different fluorescent dyes may have wavelength absorption maximums all of which differ from each other by at least about 5 nm (preferably by at least about 10 nm).

Where a fluorescent dye is used to determine the melting temperature of a nucleic acid in the method, the fluorescent dye may emit a signal that can be distinguished from a signal emitted by any other of the different fluorescent dyes that are used to label the oligonucleotides. For example, the fluorescent dye for determining the melting temperature of a nucleic acid may have a wavelength emission maximum that differs from the wavelength emission maximum of any other fluorescent dye that is used for labeling an oligonucleotide by at least about 5 nm (preferably by least about 10 nm). In some embodiments, the fluorescent dye for determining the melting temperature of a nucleic acid may be excited by different wavelength energy than any other of the different fluorescent dyes that are used to label the oligonucleotides. For example, the fluorescent dye for determining the melting temperature of a nucleic acid may have a wavelength absorption maximum that differs from the wavelength absorption maximum of any fluorescent dye that is used for labeling an oligonucleotide by at least about 5 nm (preferably by least about 10 nm).

The methods may include determining the melting temperature of at least one nucleic acid in a sample (e.g., an amplicon), which may be used to identify the nucleic acid. Determining the melting temperature may include exposing an amplicon to a temperature gradient and observing a detectable signal from a fluorophore. Optionally, where the oligonucleotides of the method are labeled with a first fluorescent dye, determining the melting temperature of the detected nucleic acid may include observing a signal from a second fluorescent dye that is different from the first fluorescent dye. In some embodiments, the second fluorescent dye for determining the melting temperature of the detected nucleic acid is an intercalating agent. Suitable intercalating agents may include, but are not limited to SYBR™ Green 1 dye, SYBR dyes, Pico Green, SYTO dyes, SYTOX dyes, ethidium bromide, ethidium homodimer-1, ethidium homodimer-2, ethidium derivatives, acridine, acridine orange, acridine derivatives, ethidium-acridine heterodimer, ethidium monoazide, propidium iodide, cyanine monomers, 7-aminoactinomycin D, YOYO-1, TOTO-1, YOYO-3, TOTO-3, POPO-1, BOBO-1, POPO-3, BOBO-3, LOLO-1, JOJO-1, cyanine dimers, YO-PRO-1, TO-PRO-1, YO-PRO-3, TO-PRO-3, TO-PRO-5, PO-PRO-1, BO-PRO-1, PO-PRO-3, BO-PRO-3, LO-PRO-1, JO-PRO-1, and mixture thereof. In suitable embodiments, the selected intercalating agent is SYBR™ Green 1 dye.

Typically, an intercalating agent used in the method will exhibit a change in fluorescence when intercalated in double-stranded nucleic acid. A change in fluorescence may include an increase in fluorescence intensity or a decrease in fluorescence intensity. For example, the intercalating agent may exhibit an increase in fluorescence when intercalated in double-stranded nucleic acid, and a decrease in fluorescence when the double-stranded nucleic acid is melted. A change in fluorescence may include a shift in fluorescence spectra (i.e., a shift to the left or a shift to the right in maximum absorbance wavelength or maximum emission wavelength). For example, the intercalating agent may emit a fluorescent signal of a first wavelength (e.g., green) when intercalated in double-stranded nucleic and emit a fluorescent signal of a second wavelength (e.g., red) when not intercalated in double-stranded nucleic acid. A change in fluorescence of an intercalating agent may be monitored at a gradient of temperatures to determine the melting temperature of the nucleic acid (where the intercalating agent exhibits a change in fluorescence when the nucleic acid melts).

In some methods, each of the amplified target nucleic acids may have different melting temperatures. For example, each of these amplified target nucleic acids may have a melting temperature that differs by at least about 1° C., more preferably by at least about 2° C., or even more preferably by at least about 4° C. from the melting temperature of any of the other amplified target nucleic acids.

The methods disclosed herein may include transcription of RNA to DNA (i.e., reverse transcription). For example, reverse transcription may be performed prior to amplification.

As used herein, “labels” or “reporter molecules” are chemical or biochemical moieties useful for labeling a nucleic acid, amino acid, or antibody. “Labels” and “reporter molecules” include fluorescent agents, chemiluminescent agents, chromogenic agents, quenching agents, radionuclides, enzymes, substrates, cofactors, inhibitors, magnetic particles, electrochemiluminescent labels, such as ORI-TAG™ (Igen), ligands having specific binding partners, or any other labels that can interact with each other to enhance, alter, or diminish a signal. “Labels” or “reporter molecules” are capable of generating a measurable signal and may be covalently or noncovalently joined to an oligonucleotide. It is understood that, should the PCR be practiced using a thermocycler instrument, a label should be selected to survive the temperature cycling required in this automated process. and other moieties known in the art.

As used herein, a “fluorescent dye” or a “fluorophore” is a chemical group that can be excited by light to emit fluorescence. Some suitable fluorophores may be excited by light to emit phosphorescence. Dyes may include acceptor dyes that are capable of quenching a fluorescent signal from a fluorescent donor dye. Dyes that may be used in the disclosed methods include, but are not limited to, the following dyes and/or dyes sold under the following tradenames: 1,5 IAEDANS; 1,8-ANS; 4-Methylumbelliferone; 5-carboxy-2,7-dichlorofluorescein; 5-Carboxyfluorescein (5-FAM); 5-Carboxytetramethylrhodamine (5-TAMRA); 5-FAM (5-Carboxyfluorescein); 5-HAT (Hydroxy Tryptamine); 5-Hydroxy Tryptamine (HAT); 5-ROX (carboxy-X-rhodamine); 5-TAMRA (5-Carboxytetramethylrhodamine); 6-carboxy-fluorescein; 6-Carboxyrhodamine 6G; 6-CR 6G; 6-JOE; 7-Amino-4-methylcoumarin; 7-Aminoactinomycin D (7-AAD); 7-Hydroxy-4-methylcoumarin; 9-Amino-6-chloro-2-methoxyacridine; ABQ; Acid Fuchsin; ACMA (9-Amino-6-chloro-2-methoxyacridine); Acridine Orange; Acridine Red; Acridine Yellow; Acriflavin; Acriflavin Feulgen SITSA; Alexa Fluor 350™; Alexa Fluor 430™; Alexa Fluor 488™; Alexa Fluor 532™; Alexa Fluor 546™; Alexa Fluor 568™; Alexa Fluor 594™; Alexa Fluor 633™; Alexa Fluor 647™; Alexa Fluor 660™; Alexa Fluor 680™; Alizarin Complexon; Alizarin Red; Allophycocyanin (APC); AMC; AMCA-S; AMCA (Aminomethylcoumarin); AMCA-X; Aminoactinomycin D; Aminocoumarin; Aminomethylcoumarin (AMCA); Anilin Blue; Anthrocyl stearate; APC (Allophycocyanin); APC-Cy7; APTS; Astrazon Brilliant Red 4G; Astrazon Orange R; Astrazon Red 6B; Astrazon Yellow 7 GLL; Atabrine; ATTO-TAG™ CBQCA; ATTO-TAG™ FQ; Auramine; Aurophosphine G; Aurophosphine; BAO 9 (Bisaminophenyloxadiazole); Berberine Sulphate; Beta Lactamase; BFP blue shifted GFP (Y66H); Blue Fluorescent Protein; BFP/GFP FRET; Bimane; Bisbenzamide; Bisbenzimide (Hoechst); Blancophor FFG; Blancophor SV; BOBO™-1; BOBO™-3; Bodipy 492/515; Bodipy 493/503; Bodipy 500/510; Bodipy 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy FL; Bodipy FL ATP; Bodipy F1-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO™-1; BO-PRO™-3; Brilliant Sulphoflavin FF; Calcein; Calcein Blue; Calcium Crimson™; Calcium Green; Calcium Orange; Calcofluor White; Carboxy-X-rhodamine (5-ROX); Cascade Blue™; Cascade Yellow; Catecholamine; CCF2 (GeneBlazer); CFDA; CFP—Cyan Fluorescent Protein; CFP/YFP FRET; Chlorophyll; Chromomycin A; CL-NERF (Ratio Dye, pH); CMFDA; Coelenterazine f; Coelenterazine fcp; Coelenterazine h; Coelenterazine hcp; Coelenterazine ip; Coelenterazine n; Coelenterazine O; Coumarin Phalloidin; C-phycocyanine; CPM Methylcoumarin; CTC; CTC Formazan; Cy2™; Cy3.1 8; Cy3.5™; Cy3™; Cy5.1 8; Cy5.5™; Cy5™; Cy7™; Cyan GFP; cyclic AMP Fluorosensor (FiCRhR); Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3; DCFDA; DCFH (Dichlorodihydrofluorescein Diacetate); DDAO; DHR (Dihydrorhodamine 123); Di-4-ANEPPS; Di-8-ANEPPS (non-ratio); DiA (4-Di-16-ASP); Dichlorodihydrofluorescein Diacetate (DCFH); DiD—Lipophilic Tracer; DiD (DiIC18(5)); DIDS; Dihydrorhodamine 123 (DHR); DiI (DiIC18(3)); Dinitrophenol; DiO (DiOC18(3)); DiR; DiR (DiIC18(7)); DNP; Dopamine; DsRed; DTAF; DY-630-NHS; DY-635-NHS; EBFP; ECFP; EGFP; ELF 97; Eosin; Erythrosin; Erythrosin ITC; Ethidium Bromide; Ethidium homodimer-1 (EthD-1); Euchrysin; EukoLight; Europium (III) chloride; EYFP; Fast Blue; FDA; Feulgen (Pararosaniline); FITC; Flazo Orange; Fluo-3; Fluo-4; Fluorescein (FITC); Fluorescein Diacetate; Fluoro-Emerald; Fluoro-Gold (Hydroxystilbamidine); Fluor-Ruby; Fluor X; FM 1-43™; FM 4-46; Fura Red™; Fura Red™/Fluo-3; Fura-2; Fura-2/BCECF; Genacryl Brilliant Red B; Genacryl Brilliant Yellow 10GF; Genacryl Pink 3G; Genacryl Yellow 5GF; GeneBlazer (CCF2); GFP (S65T); GFP red shifted (rsGFP); GFP wild type, non-UV excitation (wtGFP); GFP wild type, UV excitation (wtGFP); GFPuv; Gloxalic Acid; Granular Blue; Haematoporphyrin; Hoechst 33258; Hoechst 33342; Hoechst 34580; HPTS; Hydroxycoumarin; Hydroxystilbamidine (FluoroGold); Hydroxytryptamine; Indo-1; Indodicarbocyanine (DiD); Indotricarbocyanine (DiR); Intrawhite Cf; JC-1; JO-JO-1; JO-PRO-1; Laurodan; LDS 751 (DNA); LDS 751 (RNA); Leucophor PAF; Leucophor SF; Leucophor WS; Lissamine Rhodamine; Lissamine Rhodamine B; Calcein/Ethidium homodimer; LOLO-1; LO-PRO-1; Lucifer Yellow; Lyso Tracker Blue; Lyso Tracker Blue-White; Lyso Tracker Green; Lyso Tracker Red; Lyso Tracker Yellow; LysoSensor Blue; LysoSensor Green; LysoSensor Yellow/Blue; Mag Green; Magdala Red (Phloxin B); Mag-Fura Red; Mag-Fura-2; Mag-Fura-5; Mag-Indo-1; Magnesium Green; Magnesium Orange; Malachite Green; Marina Blue; Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; NED™; Nitrobenzoxadidole; Noradrenaline; Nuclear Fast Red; Nuclear Yellow; Nylosan Brilliant Iavin EBG; Oregon Green; Oregon Green 488-X; Oregon Green™; Oregon Green™ 488; Oregon Green™ 500; Oregon Green™ 514; Pacific Blue; Pararosaniline (Feulgen); PBFI; PE-Cy5; PE-Cy7; PerCP; PerCP-Cy5.5; PE-TexasRed [Red 613]; Phloxin B (Magdala Red); Phorwite AR; Phorwite BKL; Phorwite Rev; Phorwite RPA; Phosphine 3R; Phycoerythrin B [PE]; Phycoerythrin R [PE]; PKH26 (Sigma); PKH67; PMIA; Pontochrome Blue Black; POPO-1; POPO-3; PO-PRO-1; PO-PRO-3; Primuline; Procion Yellow; Propidium Iodid (PI); PyMPO; Pyrene; Pyronine; Pyronine B; Pyrozal Brilliant Flavin 7GF; QSY 7; Quinacrine Mustard; Red 613 [PE-TexasRed]; Resorufin; RH 414; Rhod-2; Rhodamine; Rhodamine 110; Rhodamine 123; Rhodamine 5 GLD; Rhodamine 6G; Rhodamine B; Rhodamine B 200; Rhodamine B extra; Rhodamine BB; Rhodamine BG; Rhodamine Green; Rhodamine Phallicidine; Rhodamine Phalloidine; Rhodamine Red; Rhodamine WT; Rose Bengal; R-phycocyanine; R-phycoerythrin (PE); RsGFP; S65A; S65C; S65L; S65T; Sapphire GFP; SBFI; Serotonin; Sevron Brilliant Red 2B; Sevron Brilliant Red 4G; Sevron Brilliant Red B; Sevron Orange; Sevron Yellow L; sgBFP™; sgBFP™ (super glow BFP); sgGFP™; sgGFP™ (super glow GFP); SITS; SITS (Primuline); SITS (Stilbene Isothiosulphonic Acid); SNAFL calcein; SNAFL-1; SNAFL-2; SNARF calcein; SNARF1; Sodium Green; SpectrumAqua; SpectrumGreen; SpectrumOrange; Spectrum Red; SPQ (6-methoxy-N-(3-sulfopropyl)quinolinium); Stilbene; Sulphorhodamine B can C; Sulphorhodamine G Extra; SYTO 11; SYTO 12; SYTO 13; SYTO 14; SYTO 15; SYTO 16; SYTO 17; SYTO 18; SYTO 20; SYTO 21; SYTO 22; SYTO 23; SYTO 24; SYTO 25; SYTO 40; SYTO 41; SYTO 42; SYTO 43; SYTO 44; SYTO 45; SYTO 59; SYTO 60; SYTO 61; SYTO 62; SYTO 63; SYTO 64; SYTO 80; SYTO 81; SYTO 82; SYTO 83; SYTO 84; SYTO 85; SYTOX Blue; SYTOX Green; SYTOX Orange; TET™; Tetracycline; Tetramethylrhodamine (TRITC); Texas Red™; Texas Red-X™ conjugate; Thiadicarbocyanine (DiSC3); Thiazine Red R; Thiazole Orange; Thioflavin 5; Thioflavin S; Thioflavin TCN; Thiolyte; Thiozole Orange; Tinopol CBS (Calcofluor White); TMR; TO-PRO-1; TO-PRO-3; TO-PRO-5; TOTO-1; TOTO-3; TriColor (PE-Cy5); TRITC TetramethylRodamineIsoThioCyanate; True Blue; TruRed; Ultralite; Uranine B; Uvitex SFC; VIC®; wt GFP; WW 781; X-Rhodamine; XRITC; Xylene Orange; Y66F; Y66H; Y66W; Yellow GFP; YFP; YO-PRO-1; YO-PRO-3; YOYO-1; YOYO-3; and salts thereof.

Fluorescent dyes or fluorophores may include derivatives that have been modified to facilitate conjugation to another reactive molecule. As such, fluorescent dyes or fluorophores may include amine-reactive derivatives such as isothiocyanate derivatives and/or succinimidyl ester derivatives of the fluorophore.

The oligonucleotides and nucleotides of the disclosed methods may be labeled with a quencher. Quenching may include dynamic quenching (e.g., by FRET), static quenching, or both. Suitable quenchers may include Dabcyl. Suitable quenchers may also include dark quenchers, which may include black hole quenchers sold under the tradename “BHQ” (e.g., BHQ-0, BHQ-1, BHQ-2, and BHQ-3, Biosearch Technologies, Novato, Calif.). Dark quenchers also may include quenchers sold under the tradename “QXL™” (Anaspec, San Jose, Calif.). Dark quenchers also may include DNP-type non-fluorophores that include a 2,4-dinitrophenyl group.

In some situations, it is desirable to use two interactive labels on a single oligonucleotide with due consideration given for maintaining an appropriate spacing of the labels on the oligonucleotide to permit the separation of the labels during oligonucleotide hydrolysis. It can be similarly desirable to use two interactive labels on different oligonucleotides, such as, for example, the reporter and the second region of the second primer. In this embodiment, the reporter and the second region are designed to hybridize to each other. Again, consideration is given to maintaining an appropriate spacing of the labels between the oligonucleotides when hybridized.

One type of interactive label pair is a quencher-dye pair. Preferably, the quencher-dye pair is comprised of a fluorophore and a quencher. Suitable fluorophores are described herein and may include, but are not limited to fluorescein, cascade blue, hexachloro-fluorescein, tetrachloro-fluorescein, TAMRA, ROX, Cy3, Cy3.5, Cy5, Cy5.5, 4,4-difluoro-5,7-diphenyl-4-bora-3a,4a-diaza-s-indacene-3-propionic acid, 4,4-difluoro-5,p-methoxyphenyl-4-bora-3a,4a-diazas-indacene-3-propionic acid, 4,4-difluoro-5-styryl-4-bora-3a,4-adiaza-S-indacene-propionic acid, 6-carboxy-X-rhodamine, N,N,N′,N′-tetramethyl-6-carboxyrhodamine, Texas Red, Eosin, fluorescein, 4,4-difluoro-5,7-diphenyl-4-bora-3a,4a-diaza-s-indacene-3-propionic acid, 4,4-difluoro-5,p-ethoxyphenyl-4-bora-3a,4a-diaza-s-indacene 3-propionic acid and 4,4-difluoro-5-styryl-4-bora-3a,4a-diaza-S-indacene-propionic acid. Suitable quenchers include, for example, Dabcyl, QSY7™ (Molecular Probes, Eugene, Oreg.) and the like. In addition, dyes can also be used as a quencher if they absorb the emitted light of another dye.

The labels can be attached to the nucleotides, including non-natural bases, or oligonucleotides directly or indirectly by a variety of techniques. Depending upon the precise type of label used, the label can be located at the 5′ or 3′ end of the reporter, located internally in the reporter's nucleotide sequence, or attached to spacer arms extending from the reporter and having various sizes and compositions to facilitate signal interactions. Using commercially available phosphoramidite reagents, one can produce oligonucleotides containing functional groups (e.g., thiols or primary amines) at either terminus, for example by the coupling of a phosphoramidite dye to the 5′ hydroxyl of the 5′ base by the formation of a phosphate bond, or internally, via an appropriately protected phosphoramidite, and can label them using protocols described in, for example, PCR Protocols: A Guide to Methods and Applications, ed. by Innis et al., Academic Press, Inc., 1990, incorporated herein by reference.

Methods for incorporating oligonucleotide functionalizing reagents having one or more sulfhydryl, amino or hydroxyl moieties into the oligonucleotide reporter sequence, typically at the 5′ terminus, are described in U.S. Pat. No. 4,914,210, incorporated herein by reference. For example, a 5′ phosphate group can be incorporated as a radioisotope by using polynucleotide kinase and [γ³²P]ATP to provide a reporter group. Biotin can be added to the 5′ end by reacting an aminothymidine residue, introduced during synthesis, with an N-hydroxysuccinimide ester of biotin.

Labels at the 3′ terminus, for example, can employ polynucleotide terminal transferase to add the desired moiety, such as for example, cordycepin, ³⁵S-dATP, and biotinylated dUTP.

Oligonucleotide derivatives are also available as labels. For example, etheno-dA and etheno-A are known fluorescent adenine nucleotides which can be incorporated into a reporter. Similarly, etheno-dC is another analog that can be used in reporter synthesis. The reporters containing such nucleotide derivatives can be hydrolyzed to release much more strongly fluorescent mononucleotides by the polymerase's 5′ to 3′ nuclease activity as nucleic acid polymerase extends a primer during PCR.

In some embodiments, the labels may comprise first and second labels wherein the first label is separated from the second label by a nuclease-susceptible cleavage site.

In some embodiments, the disclosed assays are used for the detection of anthrax toxin-specific sequences. For example, the assays may utilize MultiCode®-RTx PCR technology, which is disclosed in U.S. Patent Application Publication No. 2002-0150900, incorporated herein by reference. The assays may be performed using real-time or continuous methods using any suitable commercial thermal cycler. The disclosed technology may be used to detect nucleic acid targets obtained from any source (e.g.; human, animal and infectious disease samples). Advantages of the MultiCode®-RTx system may include: high sensitivity, high specificity, rapid cycling with real-time readout, thermal melt at the end of run to verify specific amplification of target sequences, inclusion of internal RT-PCR control, excellent stability, and rapid creation of new assays based on genetic sequences.

### Illustrative Embodiments

The following illustrated embodiments are presented to aid the reader in understanding the methods and kits described herein and are not intended to be limiting.

A first illustrated embodiment includes a method for identifying a virulent bacteria in a sample comprising: (a) reacting a mixture that comprises: (i) nucleic acid isolated from the sample; (ii) at least one oligonucleotide capable of specifically hybridizing to nucleic acid of plasmid pX01; and (iii) at least one oligonucleotide capable of specifically hybridizing to nucleic acid of plasmid pX02; (b) detecting nucleic acid of plasmid pX01; and (c) detecting nucleic acid of plasmid pX02.

A second illustrated embodiment includes the method of illustrated embodiment one, wherein the virulent bacteria is a member of the Bacillus genus.

A third illustrated embodiment includes the method of illustrated embodiment one or two, wherein the virulent bacteria is a strain of Bacillus anthracis.

A fourth illustrated embodiment includes the methods of illustrated embodiments one, two or three, wherein the reaction mixture further comprises: (iv) internal control nucleic acid; and (v) at least one oligonucleotide capable of specifically hybridizing to the internal control nucleic acid; and the method further comprises: (d) detecting the internal control nucleic acid nucleic acid.

A fifth illustrated embodiment includes the methods of illustrated embodiments one, two, three or four, wherein the at least one oligonucleotide that is capable of specifically hybridizing to nucleic acid of plasmid pX01 is capable of specifically hybridizing to at least one of nucleic acid of cya (edema factor), nucleic acid of lef (lethal factor), nucleic acid of pagA (protective antigen), nucleic acid of atxA, and nucleic acid of pagR.

A sixth illustrated embodiment includes the method of illustrated embodiment five, wherein the at least one oligonucleotide is capable of specifically hybridizing to nucleic acid of cya (edema factor) or pagA (protective antigen).

A seventh illustrated embodiment includes the methods of any of illustrated embodiments one through six, wherein the at least one oligonucleotide that is capable of specifically hybridizing to nucleic acid of plasmid pX02 is capable of specifically hybridizing to at least one of nucleic acid of capB, nucleic acid of cap C, nucleic acid of capA, nucleic acid of dep, and nucleic acid of acpA.

An eighth illustrated embodiment includes the method of illustrated embodiment seven, wherein the at least one oligonucleotide is capable of specifically hybridizing to nucleic acid of capB.

A ninth illustrated embodiment includes the methods of any one of illustrated embodiments one through eight, wherein the reaction mixture comprises at least two oligonucleotides capable of specifically hybridizing to nucleic acid of plasmid pX01 and the method further comprises amplifying nucleic acid of plasmid pX01 using the two oligonucleotides as primers.

A tenth illustrated embodiment includes the methods of any one of illustrated embodiments one through nine, wherein the reaction mixture comprises at least two oligonucleotides capable of specifically hybridizing to nucleic acid of plasmid pX02 and the method further comprises amplifying nucleic acid of plasmid pX02 using the two oligonucleotides as primers.

An eleventh illustrated embodiment includes the methods of any one of illustrated embodiments four through ten, wherein the reaction mixture comprises at least two oligonucleotides capable of specifically hybridizing to the control nucleic acid and the method further comprises amplifying the control nucleic acid using the two oligonucleotides as primers.

A twelfth illustrated embodiment includes the method of illustrated embodiment nine, wherein at least one of the two oligonucleotides used as primers includes a label.

A thirteenth illustrated embodiment includes the method of illustrated embodiment nine, wherein at least one of the two oligonucleotides used as primers includes at least one nucleotide other than A, C, G, T, and U.

A fourteenth illustrated embodiment includes the method of illustrated embodiment thirteen, wherein the nucleotide other than A, C, G, T, and U, is selected from iC and iG.

A fifteenth illustrated embodiment includes the method of any one of illustrated embodiments twelve through fourteen, wherein the label comprises a fluorophore and the reaction mixture further comprises a nucleotide covalently linked to a quencher.

A sixteenth illustrated embodiment includes the method of illustrated embodiment fifteen, wherein the nucleotide covalently linked to the quencher comprises iC or iG.

A seventeenth illustrated embodiment includes the method of illustrated embodiment ten, wherein at least one of the two oligonucleotides used as primers includes a label.

An eighteenth illustrated embodiment includes the method of illustrated embodiment ten, wherein at least one of the two oligonucleotides used as primers includes at least one nucleotide other than A, C, G, T, and U.

A nineteenth illustrated embodiment includes the method of illustrated embodiment eighteen, wherein the nucleotide other than A, C, G, T, and U, is selected from iC and iG.

A twentieth illustrated embodiment includes the method of any one of illustrated embodiments seventeen through nineteen, wherein the label comprises a fluorophore and the reaction mixture further comprises a nucleotide covalently linked to a quencher.

A twenty-first illustrated embodiment includes the method of illustrated embodiment twenty, wherein the nucleotide covalently linked to the quencher comprises iC or iG.

A twenty-second illustrated embodiment includes the method of illustrated embodiment eleven, wherein at least one of the two oligonucleotides used as primers includes a label.

A twenty-third illustrated embodiment includes the method of illustrated embodiment eleven, wherein at least one of the two oligonucleotides used as primers includes at least one nucleotide other than A, C, G, T, and U.

A twenty-fourth illustrated embodiment includes the method of illustrated embodiment twenty-three, wherein the nucleotide other than A, C, G, T, and U, is selected from iC and iG.

A twenty-fifth illustrated embodiment includes the method of any one of illustrated embodiments twenty-two to twenty-four, wherein the label comprises a fluorophore and the reaction mixture further comprises a nucleotide covalently linked to a quencher.

A twenty-sixth illustrated embodiment includes the method of illustrated embodiment twenty-five, wherein the nucleotide covalently linked to the quencher comprises iC or iG.

A twenty-seventh illustrated embodiment includes the method of any one of illustrated embodiments one to twenty-six, wherein the nucleic acid of plasmid pX01 is present on a plasmid in the bacteria or present within the genome of the bacteria.

A twenty-eighth illustrated embodiment includes the method of any one of illustrated embodiments one to twenty-seven, wherein the nucleic acid of plasmid pX02 is present on a plasmid in the bacteria or present within the genome of the bacteria.

A twenty-ninth illustrated embodiment includes a method for detecting a virulent bacteria in a sample comprising: (a) reacting a mixture that comprises: (i) nucleic acid isolated from the sample; (ii) a first pair of oligonucleotides capable of specifically hybridizing to nucleic acid of plasmid pX01, wherein at least one oligonucleotide of the first pair includes a first label; (iii) a second pair of oligonucleotides capable of specifically hybridizing to nucleic acid of plasmid pX02, wherein at least one oligonucleotide of the second pair includes a second label; (iv) control nucleic acid; and (v) a third pair of oligonucleotides capable of specifically hybridizing to the control nucleic acid, wherein at least one oligonucleotide of the third pair includes a third label; and the first label, second label, and third label are different; and (b) amplifying and detecting: (i) the nucleic acid of plasmid pX01; (ii) nucleic acid of plasmid pX02, and (iii) the control nucleic acid.

A thirtieth illustrated embodiment includes the method of illustrated embodiment twenty-nine, wherein the virulent bacteria is a member of the Bacillus genus.

A thirty-first illustrated embodiment includes the method of illustrated embodiment thirty, wherein the virulent bacteria is a strain of Bacillus anthracis.

A thirty-second illustrated embodiment includes the method of any one of illustrated embodiments twenty-nine to thirty-one, wherein the first pair of oligonucleotides is capable of specifically hybridizing to nucleic acid selected from nucleic acid of cya (edema factor), nucleic acid of lef (lethal factor), nucleic acid of pagA (protective antigen), nucleic acid of atxA, and nucleic acid of pagR.

A thirty-third illustrated embodiment includes the method of illustrated embodiment thirty-two, wherein the first pair of oligonucleotides is capable of specifically hybridizing to nucleic acid of cya (edema factor) or nucleic acid of pagA (protective antigen).

A thirty-fourth illustrated embodiment includes the method of any one of illustrated embodiments twenty-nine to thirty-three, wherein the second pair of oligonucleotides is capable of specifically hybridizing to nucleic acid of capB, nucleic acid of capC, nucleic acid of capA, nucleic acid of dep, and nucleic acid of acpA.

A thirty-fifth illustrated embodiment includes the method of illustrated embodiment thirty-four, wherein the second pair of oligonucleotides is capable of specifically hybridizing to nucleic acid of capB.

A thirty-sixth illustrated embodiment includes the method of illustrated embodiment twenty-nine, wherein at least one oligonucleotide of the first, second, and third pair of oligonucleotides includes at least one nucleotide other than A, C, G, T, and U.

A thirty-seventh illustrated embodiment includes the method of illustrated embodiment thirty-six, wherein the nucleotide other than A, C, G, T, and U, is selected from iC and iG.

A thirty-eighth illustrated embodiment includes the method of any one of illustrated embodiments twenty-nine to thirty-seven, wherein the first label, second label, and third label comprise three different fluorophores and the reaction mixture further comprises a nucleotide covalently linked to a quencher capable of quenching the three different fluorophores.

A thirty-ninth illustrated embodiment includes the method of any one of illustrated embodiments twenty-nine to thirty-eight further comprising: (d) determining a melting temperature for amplified nucleic acid of at least one of amplified nucleic acid of plasmid pX01, amplified nucleic acid of plasmid pX02, and amplified control nucleic acid.

A fortieth illustrated embodiment includes the method of any one of illustrated embodiments twenty-nine to thirty-nine, wherein the nucleic acid of plasmid pX01 is present on a plasmid in the bacteria or present within the genome of the bacteria.

A forty-first illustrated embodiment includes the method of any one of illustrated embodiments twenty-nine to forty, wherein the nucleic acid of plasmid pX02 is present on a plasmid in the bacteria or present within the genome of the bacteria.

A forty-second illustrated embodiment includes a kit for performing any of the methods of illustrated embodiments one through forty-one.

A forty-third illustrated embodiment includes the kit of illustrated embodiment forty-two, comprising: (a) a first pair of oligonucleotides capable of specifically hybridizing to nucleic acid of plasmid pX01, wherein at least one oligonucleotide of the first pair includes a first label; and (b) a second pair of oligonucleotides capable of specifically hybridizing to nucleic acid of plasmid pX02, wherein at least one oligonucleotide of the second pair includes a second label; wherein the first label and second label are different.

A forty-fourth illustrated embodiment includes the kit of illustrated embodiment forty-three further comprising: (c) control nucleic acid; and (d) a third pair of oligonucleotides capable of specifically hybridizing to the control nucleic acid, wherein at least one oligonucleotide of the third pair includes a third label; wherein the first label, second label, and third label are different.

A forty-fifth illustrated embodiment includes the kit of illustrated embodiment forty-three or forty-four, wherein the first pair of oligonucleotides is capable of specifically hybridizing to nucleic acid selected from nucleic acid of cya (edema factor), nucleic acid of lef (lethal factor), nucleic acid of pagA (protective antigen), nucleic acid of atxA, and nucleic acid of pagR.

A forty-sixth illustrated embodiment includes the kit of illustrated embodiment forty-five, wherein the first pair of oligonucleotides is capable of specifically hybridizing to nucleic acid selected from nucleic acid of cya (edema factor) or nucleic acid of pagA (protective antigen).

A forty-seventh illustrated embodiment includes any of the kits of illustrated embodiments forty-three through forty-six, wherein the second pair of oligonucleotides is capable of specifically hybridizing to nucleic acid selected from nucleic acid of capB, nucleic acid of capC, nucleic acid of capA, nucleic acid of dep, and nucleic acid of acpA.

A forty-eighth illustrated embodiment includes the kit of illustrated embodiment forty-seven, wherein the second pair of oligonucleotides is capable of specifically hybridizing to nucleic acid of capB.

A forty-ninth illustrated embodiment includes any of the kits of illustrated embodiments forty-three through forty-eight, wherein at least one oligonucleotide of the first, second, and third pair of oligonucleotides includes at least one nucleotide other than A, C, G, T, and U.

A fiftieth illustrated embodiment includes the kit of illustrated embodiment forty-nine, wherein the nucleotide other than A, C, G, T, and U, is selected from iC and iG.

A fifty-first illustrated embodiment includes any of the kits of illustrated embodiments forty-three through fifty, wherein the first label, second label, and third label comprise three different fluorophores and the kit further comprises a nucleotide covalently linked to a quencher capable of quenching the three different fluorophores.

A fifty-second illustrated embodiment includes any of the kits of illustrated embodiments forty-three through fifty-one, further comprising a reagent for determining a melting temperature of nucleic acid.

### EXAMPLES

The methods disclosed herein may be performed according to the following Example. Kits as disclosed herein may include one or more components described in the Example.

Traditionally, Bacillus anthracis has been distinguished from other members of the B. cereus group by time-consuming techniques such as colony morphology, penicillin susceptibility, gamma phage susceptibility, lack of hemolysis, and motility. These methods are giving way to more rapid and quantifiable nucleic acid-based assays. Since the publication of the polymerase chain reaction (PCR) in 1987, applications involving this technology have revolutionized molecular medicine. More recently, real-time PCR is becoming a preferred approach. This is mainly due to the intrinsic benefits of real-time PCR such as quantitative accuracy, single copy sensitivity, high level of specificity and speed. Additionally, real-time PCR can be multiplexed to allow for multiple target analysis in a single reaction. As with the case of anthrax toxin gene detection, multiplexing is clearly beneficial as there are two virulent plasmids (pX01 and pX02) required for full virulence.

Recently, the presence of a plasmid in a strain of B. cereus with a 99.6% homology to a toxin encoding plasmid found in B. anthracis indicates that genetic diagnosis is more complicated than once thought. Genes specifically associated with inhalation anthrax are located on two plasmids, pX01 and pX02. The 182-kb pX01 plasmid harbors the structural genes for the anthrax toxin proteins [cya (edema factor), lef (lethal factor), and pagA (protective antigen)], as well as two trans-acting regulatory genes (atxA and pagR). The 96-kb pX02 plasmid carries three genes required for capsule synthesis (capB, capC, and capA), a gene associated with capsule degradation (dep), and a trans-acting regulatory gene (acpA). A high degree of sequence conservation was shown between plasmid pX01 and the chromosome of some members of the B. cereus group, with several strains showing 80 to 98% homology. Additionally, a high-coverage draft genome sequence of a B. cereus isolate (G9241) revealed the presence of a circular plasmid, named pBCX01, with 99.6% similarity with the B. anthracis toxin-encoding plasmid, pX01. In addition, this isolate was found to be 100% lethal in mice with symptoms similar to inhalation anthrax. Therefore, a simplified multiplexed chemistry that specifically detects these plasmids, or genes associated with these plasmids, may prove to be as or more important than identification of the organism itself.

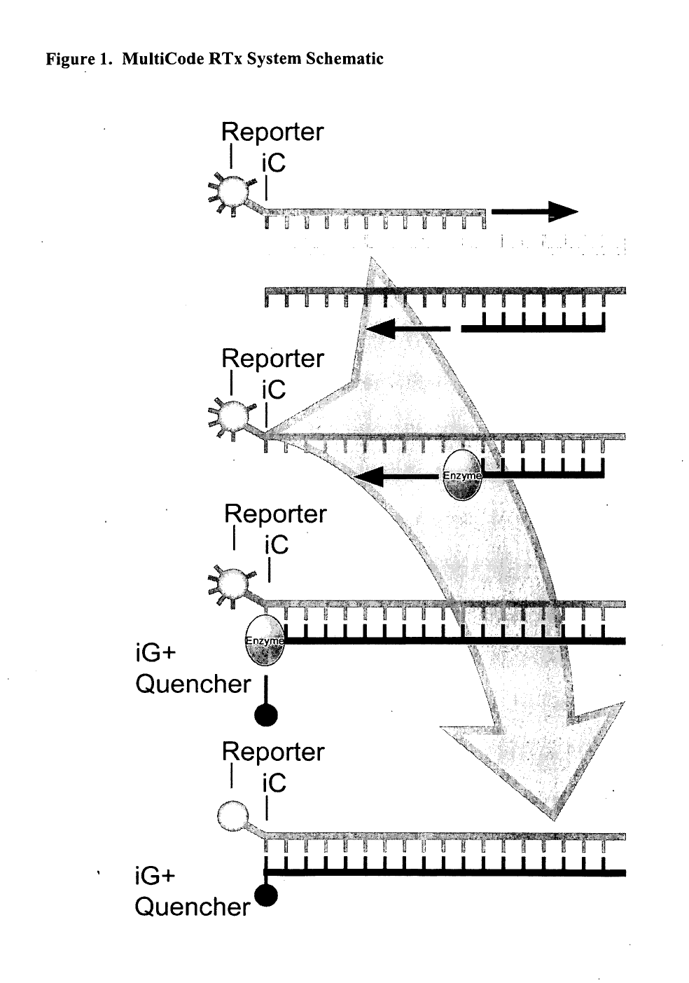

To this end, two triplex assays using the MultiCode®-RTx platform were developed. MultiCode-RTx uses an expanded genetic base pair constructed from 2′-deoxy-5-methyl-isocytidine (iC) and 2′-deoxy-isoguanosine (iG). In natural DNA, two complementary strands are joined by a sequence of Watson-Crick base pairs using the four standard nucleotides A, G, C and T. Yet the DNA alphabet need not be limited to the four standard nucleotides known in nature. In fact, expanded nucleobase pairs have been chemically produced. In particular, the chemistries to produce phosphoramidite and triphosphate reagents of iC and iG have been optimized and are now commercially available. The MultiCode-RTx assay uses iC and iG to site-specifically incorporate a quencher in close proximity to a fluorescent molecule during PCR (FIG. 1). Prior to running RTx, target specific forward PCR primers carrying single iC bases near distinct 5′ fluorescent reporters and standard reverse primers are constructed using standard oligonucleotide chemical synthesis. Using a commercially available reaction mix containing iGTP-Dabcyl, iC directs specific enzymatic incorporation of the iGTP-dabcyl in close proximity to each fluorophore. This incorporation reduces the fluorescence of reporters attached to the extended primers and is monitored using standard real-time PCR instrumentation. As the reaction proceeds, the instrument collects data (each target is analyzed using a distinct fluorophore and data collected in distinct channels). As more and more of the labeled primers are used up, the fluorescence signal specific for that primer goes down. As with all other real-time chemistries, standard curves constructed from Ct data from known concentrations of each target are used to determine concentrations within unknown samples. Additionally, the reaction can be analyzed for correct product formation after cycling is complete by melting the amplicons and determining their melting temperatures. This melt analysis can be used to verify that the anticipated amplicon was created.

Using this chemistry, two three-color LightCycler-1 multiplex real-time PCR assays have been developed. The LightCycler-1 is an instrument with a signal excitation laser and optics identical to the Idaho Technology R.A.P.I.D. (Ruggedized Advanced Pathogen Identification Device), acquired through the Joint Biological Agent Identification and Diagnostic System (JBAIDS) as the single Department of Defense accepted platform for both identification and diagnostic confirmation of biological agents.

The first assay is specific for pagA:capB:IPC (IPC=Internal Positive Control) and the second is specific for cya:capB:IPC; both assays are specific for genes associated with inhalation anthrax. Each triplex RTx assay had an analytical detection limit of one to nine plasmid copy equivalents and 100% analytical specificity with a 95% confidence interval width (CI) of 9% and 100% analytical sensitivity with a CI of 2%. Thus, the two different RTx systems demonstrated high sensitivity and specificity with limits of detection nearing single copy levels. The assays are able to specifically differentiate these targets from multiple other Bacillus species with limits of detection at or below previously published singleplex assays.

### 1. Materials and Methods

Bacterial growth and extraction: The bacterial strains analyzed in this study were acquired from the American Type Culture Collection (Manassas, Va.), clinics, or entries from previous U.S. Army Medical Research Institute of Infectious Diseases (Fort Detrick, Frederick, Md.) collections. Either Bactozol kits (Molecular Research Center, Inc., Cincinnati, Ohio) or QIAamp DNA minikits (Qiagen, Valencia, Calif.) were used to extract DNA. Bactozol kits were used in accordance with the manufacturer's recommendations. QIAamp kits were used as follows. Cells were pelleted and resuspended in 180 μl of Dulbecco's phosphate-buffered saline (GibcoBRL, Rockville, Md.). Twenty microliters of proteinase K and 200 μl of AL buffer (Qiagen) were added and mixed by vortexing. The mixture was incubated for 60 min at 55° C. to lyse the cells. After incubation, 210 μl of 100% ethanol was added to the sample. The mixture was subject to RNAse digestion and transferred to a QIAamp spin column and centrifuged at 6,000×g for 2 min. Next, 500 μl of AW1 buffer (Qiagen) was added to the column, and the sample was centrifuged for 2 min at 6,000×g. Following this centrifugation step, 500 μl of AW2 buffer (Qiagen) was added to the column, and the sample was centrifuged at 6,000×g for 2 min. Finally, 100 μl of AE buffer (Qiagen) preheated to 70° C. was applied to the column, and the sample was centrifuged at 6,000×g for 1 min to elute the DNA. The DNA concentration was determined by measuring the absorptivity of each sample at 260 nm with a DU series 500 spectrometer (Beckman Instruments, Fullerton, Calif.).

Primers: All primer designations, sequence make-up, design software implemented, and concentrations used can be found in Tables 1 and 4. Oligonucletides used in the assays disclosed herein were designed based on the reference anthrax genome sequence deposited in GenBank. See Table 2 for strain numbers. Primer design packages used for this study were Primer Express (Applied Biosystems, Foster City, Calif.), Primer3 (12) and Visual OMP (DNA Software, Inc., Ann Arbor, Mich.). Primers AS005 through 008 were initially designed for Taqman use. Incorporation of the iC (X) nucleotides during synthesis were done using standard coupling conditions. All synthetic DNAs were quantitated by using extinction coefficients corresponding to the nucleotide makeup and examining initial stocks by OD 260. The DNAs were diluted to appropriate working concentrations in 10 mM MOPS and 0.1 mM EDTA. BLASTN searches were performed for all primers and probes to eliminate priming to sequences other than those specified. All oligonucleotides were manufactured and purified by IDT (Coralville, Iowa). Both cya and pagA specific primer pair sets have a 100% match to B. cereus isolate G9241 pBCX01 plasmid DNA. The capB primer pairs are not complementary to any known sequence within the G9241 isolate.

Real-time PCR amplification: PCR conditions were 1× ISOlution™ 1147 buffer (PN 1147 EraGen, Madison, Wis.) with addition of 2 mM MgCl₂ to reach a final concentration of 4 mM MgCl₂ per reaction, at a volume of 25 μl. PCR primers used and their concentrations can be found in Table 1. (See also Table 4 for SEQ ID NOs). Titanium Taq DNA polymerase (Clontech, Palo Alto, Calif.) was used at 1× concentration. Cycling parameters for the two triplex assays were 2 minutes denaturation at 95° C. followed by 45 cycles of 5 sec @ 95° C. denaturation, 5 sec anneal @ 55° C. (pagA:capB:IPC) or 60° C. (cya:capB:IPC); 20 sec @72° C. with optical read on the LightCycler-1 real-time thermal cycler (Roche Applied Science, Indianapolis, Ind.). Thermal melts from 60 to 95° C., 0.4° C. STEP with optical read were performed directly following the cycling.

Color Compensation: Color compensation is required for multi-color analysis on the LightCycler-1 instrument. A single compensation file could be used to correct data sets acquired from multiple instruments. This is performed by analyzing the contribution of each single type of labeled DNA oligonucleotides to the signal obtained in each of the three detection channels of the LightCycler-1. The fluorophore set (FAM, HEX, Cy5) is not employed by the standard color compensation reagents supplied by the instrument manufacturer. To compensate, solutions of oligonucleotides labeled with these dyes were used at the concentrations used in the standard compensation reagents. The instrument manufacturers compensation instructions were then followed to obtain compensation data capable of correcting for the spectral properties of our dye set.

Testing parameters: All developed assays included the detection of an IPC (DM155) that was added at a level of 1000 copies per reaction and detected with primers 1139 and 1140. The fluorescence change of IPC reaction was monitored in the F3 channel (690-730 nm) of the LightCycler-1 instrument. Performance of the IPC reaction was analyzed by determining the mean Ct, standard deviation (SD) and percent coefficient of variation (% CV) for 218 total reactions each for both of the final triplex assays.

Synthetic oligonucleotide targets corresponding to the anthrax toxin specific plasmid-associated gene targets were used to develop our assays. Standard curves (Ct vs. copy number) were constructed from runs using ten-fold dilution series of these synthetic targets from 3 to 3×10⁵ copies per reaction. Analytical specificity (true negatives/true negatives plus false positives) and sensitivity testing (true positives/true positive plus false negatives) was conducted using 100 pg of total extracted DNA from 38 strains of B. anthracis, 34 strains of B. cereus, and 13 strains of B. thuringiensis, one strain each of 4 other Bacillus sp., as well as a cross-reactivity panel consisting of 72 different strains of other bacterial species. See Table 2. Some B. anthracis strains contained copies of only one of the two anthrax toxin specific plasmids. Each 32 capillary LightCycler-1 run included at least one reaction where a positive control of 1 pg of extracted B. anthracis Ames DNA was added and at least one reaction where no target was added. The analytical limit of detection and limit of quantitation were determined by analyzing (in duplicate) serial 10-fold dilutions of extracted DNA from the Ames strain of B. anthracis starting at 1 pg and ending at 1 fg.

Analysis Software: Commercially-available real-time thermal cyclers use software designed to analyze reactions where fluorescence increases with PCR product accumulation. To analyze decreasing fluorescence results, analysis software was developed that imports RTx raw data and performs cycle threshold and melt curve analyses. Raw F1, F2, and F3 component fluorescence data for both amplification and melt programs were exported from the LightCycler-1 Analysis software (Version 5.32) as text files and analyzed with EraGen Real-time Run Importer and Analysis Desktop v0.9.8 alpha (EraGen Biosciences, Inc., Madison, Wis.).

Target Selection Criteria and Primers: Targets are selected using BLAST analysis of the anthrax plasmid encoded toxin sequence. A non-complementary region from by 1-3150 is selected and primers are designed. Three sets of primers are selected and tested in a duplex assay with an internal control system that includes an internal control target and an internal control target primer set. A system is designed such that few or no primer dimers are observed after 50 cycles of PCR.

Components: The following components are utilized: DNA Polymerase: A suitable DNA polymerase is Titanium Taq Polymerase (100 μl) (Clontech cat#8434-1) 50×, final concentration 1× (200 reactions); DNA Internal Control: One tube of 100 μl Internal Control RDNA (100 reactions); and Nuclease Free Water: One tube of 1 ml nuclease free water.

Assay Setup: For each sample to be run, the total reaction mix may be formulated according to Table 5. Total Reaction Size: 25 μL (20 μL Reaction Mix, 5 μL Target)

Reaction Procedure: Reaction mixtures are prepared on ice. Components are thawed and full resuspension of 2× Reaction Buffer is confirmed. Gentle warming by hand is performed if precipitate remains in 2× Reaction Buffer after thawing. Thawed reagents are vortexed.

Reaction mixture are prepared by mixing appropriate volumes of 2× Reaction Buffer, MgCl₂, and Nuclease Free Water. Titanium Taq is added to the mixtures. The mixtures are vortexed and incubated on ice for an additional minute. Fifty × (50×) Primer Mix and Internal Control DNA are added and the mixtures are vortexed thoroughly. Generally, internal control DNA is added to all reaction mixtures. Twenty microliters (20 μL) of reaction mix is added to each reaction tube. Five microliters (5 μL) of Dilution Buffer is added to “no target” sample wells or 5 μL of target is added to sample wells. Reaction tubes or plates are spun at ˜2000 rpm. Tubes are inserted into instrument and run.

Thermocycling Parameters: Exemplary conditions for PCR are as follows.

### Stage 1

95° C./120 Seconds

### Stage 2

95° C./5 Seconds

55° C./5 Seconds

74° C./20 Seconds (Optical Reading)

Stage 3 (repeated 45 times)

60° C./15 Seconds

### Stage 4

Start Temp=60° C.

End Temp=95° C.
Increment=0.2° C./Second

### 2. Results

Initial studies focused first on four duplex assays (target plus IPC), two specific for capB using primer pairs 1141/1142 and 007/008, one specific for cya using primer pair 1143/1144 and the fourth specific for pagA using primer pairs 005/006. Standard curves (log copy number vs Ct) constructed from assays using a series of synthetic target dilutions were linear down to 3 copies for all duplexed systems (data not shown). With over 100 reactions performed, 20 copies of synthetic DNA matching the correct gene target regions were detected 100% of the time. Primer sets were combined to develop two triplex assays; pagA:capB:IPC and cya:capB:IPC using primer sets 005-008 and 1141-1144 respectively. After cycling parameters were optimized using synthetic targets in order to reach detection levels observed for the duplex assays, analytical specificity of the two triplex assays was tested using DNA extracted from our panel of organisms. See Table 2. In this set of experiments, product formation was observed from the pagA and capB primer sets in samples that contained DNA extracted from organisms other than B. anthracis. The unidentified products differed in Tₘ from positive control based on melt analysis data suggesting template independent amplification.

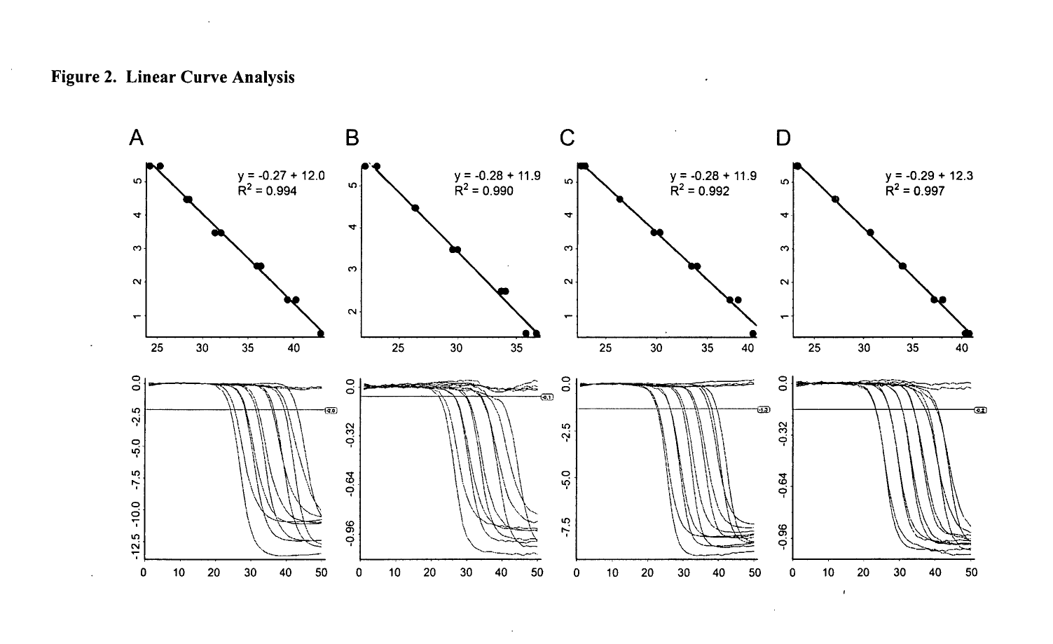

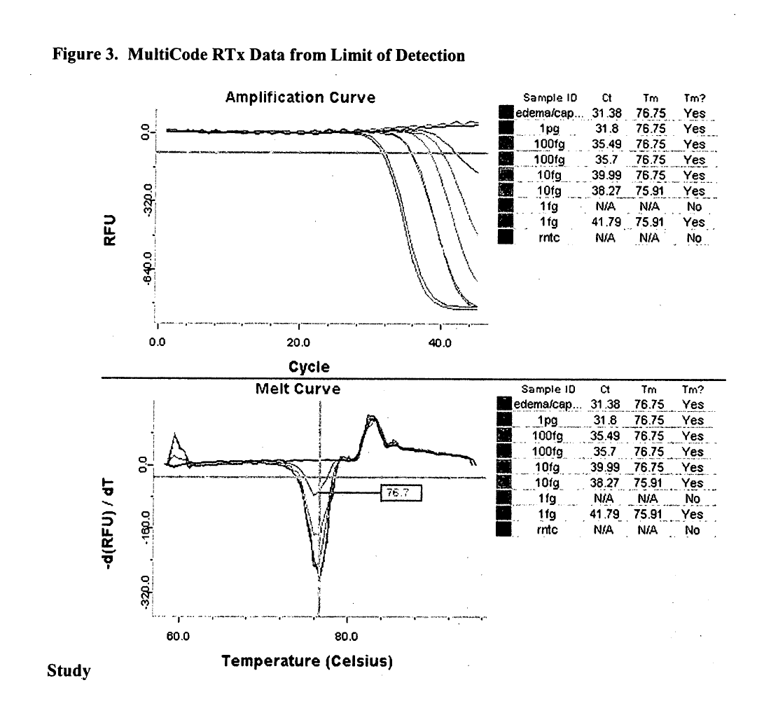

To address the observed lack of specificity, new pagA and capB primer sets were designed using Visual OMP multiplex design software. Design parameters included multiplex compatibility with the IPC and the cya primer sets. The new designs pagA:capB:IPC (containing primers 1323, 1324, 698 and 699) and cya:capB:IPC (containing primers 1143, 1144, 698 and 699) demonstrated noticeable improvements. Like the original triplexes, standard curves for the new systems were linear down to 3 copies with R² values greater than 0.99 (FIG. 2). Ten-fold serial dilutions of DNA extracted from B. anthracis Ames were made to determine the limit of detection. The extracted DNA was tested in duplicate to determine the lowest concentration that could be detected. The results indicated that the pagA:capB:IPC system was able to detect 100 fg of total extracted DNA from all replicates in both channels. The cya:capB:IPC system displayed a different set of results in that the cya specific channel was able to detect 10 fg of genomic DNA in duplicate runs (FIG. 3). Using the Ct's observed and fitting them into the standard curve equations determined above; we estimated the detection limit for the pX01 and pX02 plasmids to be 2 and 1 for the pagA:capB:IPC system and 9 and 2 copies for the cya:capB:IPC system respectively.

Unlike the original triplex systems, these new triplex systems were specific and sensitive for the target panel. For example, the pagA:capB:IPC demonstrated specificity for strains that contain only one of the two virulence plasmids (pX01 or pX02). Of the seven strains containing only pX01, only the pagA primer specific channel reported fluorescent change. Of the two strains containing only pX02, only the capB primer specific channel reported fluorescent change. Two unrelated strains (Yersinia frederiksenii and Salmonella choleraesius) displayed weak signal change. When these wells were considered to be true false positives by Ct values alone, the assay showed a ˜97% specificity. However, by including the criteria of correct melt Tm values, software analysis indicated these to be true negatives. Triplicate re-testing for both the Y. frederiksenii and the S. choleraesius samples showed no detectable product formation. Therefore, using dual criteria of Ct and correct melt Tm, the pagA:capB:IPC design was 100% specific. The total of 123 reactions testing panel DNAs from strains other than B. anthracis resulted in a 95% confidence interval width (CI) of 2%. Additionally, the pagA:capB:IPC correctly detected all 38 B. anthracis strains resulting in an analytical sensitivity of 100% with a CI of 9%. The cya:capB:IPC design also correctly detected all strains of B. anthracis including those with single plasmids, again resulting in an analytical sensitivity of 100%, CI 9%. In addition, signal change was not observed when DNAs from the panel set were added which includes no cross reactivity to the B. cereus or B. thuringiensis strains tested. The common IPC sequence amplified almost identically in all assays with mean Ct values of 33.2 and 33.5 cycles for the cya:capB:IPC and pagA:capB:IPC respectively. The SD of 0.5 cycles and 1.6% CV were identical for the two IPC reactions.

To determine the variation from run to run during the course of this study, the data from eight positive control reactions were analyzed over the course of four weeks for both triplex systems. A positive control reaction consisting of 1 pg of total extracted DNA from B. anthracis Ames was included in each LightCycler-1 carousel of 32 capillaries. Mean Cₜ and melt Tₘ, SD and % CV values from these runs are presented in Table 3. Variation in Cₜ values was greater than that of Tₘ with % CV ranging from 1.9-5.0% and 0.1-0.5% respectively.

The standard, curves from the positive control data set shown in FIG. 2 were used to estimate the copy number of each target by using the average Cₜ from the genomic DNA positive controls. It was estimated that there are about 100 copies of the pagA, capB targets in 1 pg genomic DNA, consistent with the observed limit of detection of 100 fg or about 10 copies for these targets. The cya specific assay indicates a higher copy number of around 900 copies per pg which agrees with the cya limit of detection of 10 fg or about 9 copies.

### 3. Discussion

Since 2001, when letters containing highly processed anthrax spores from the Ames strain of Bacillus anthracis were found addressed to members of congress and the media, public health diagnostic labs around the United States have become equipped with real-time PCR instruments and associated testing kits used to assay for the presence of anthrax. Real-time PCR has been chosen as the prime screening method for rapid identification due to its intrinsic benefits such as enhanced sensitivity and shortened analytical turnaround times when compared to the more standard culturing techniques.

The MultiCode-RTx triplex designs presented herein may allow for an alternative to the single-plex anthrax specific assays now employed at many of the public health labs. The RTx triplex systems developed reliably detected 10-100 fg of total B. anthracis extracted DNA. These amounts translated into a copy number limit of detection of 1-9 anthrax toxin specific plasmids. Virulence plasmids in B. anthracis may be found at copies higher than 1 per genome which would further improve the limit of detection. Analytical specificity and sensitivity were comparable to reported singleplex real-time assays.

The data presented herein also show for the first time simultaneous quantitative detection of three independent targets using three colors on the LightCycler-1. Previous usage of three-color detection on the LightCycler was used for genotyping via melt analysis. Unlike this multicolor system and many other real-time PCR chemistries, RTx does not use probes. There are perceived benefits to using probes in PCR real-time detection, with the most important being specificity. Yet, probe based systems are clearly more difficult to design and are complicated by the inherent fact that single-stranded DNA targets form intra-molecular structures that interfere with probe binding. Many primer design software programs have been developed to compensate for this by focusing on the probe region and probe design, while relaxing primer restraints. When proper primer design software is used, probes are not needed for specificity as the data demonstrates. There are other real-time PCR technologies that do not use probes. Compared to these systems, RTx does not require incorporation of hairpins in the primer design nor does it require special base sequence make-up near the 3′ ends. This allows for easy use of previously designed primer pairs. The RTx technology also allows multiplexing in order to assay multiple targets or to include internal controls. Real time multiplexing is not an option with SYBR Green, though post reaction melt analysis multiplexing may be implemented.

For bioweapon detection, the demonstration using the LightCycler-1 instrument was important because it is essentially the same instrument as the R.A.P.I.D. For this reason, the successful multiplex results using the LightCycler-1 suggest that the RTx system would work equally well on the R.A.P.I.D. system. This device was recently chosen by the U.S. Army Space and Missile Defense Command Joint Biological Agent Identification and Diagnostic System (JBAIDS) for biothreat sample processing. The R.A.P.I.D. is a specialty instrument for military field hospitals, first responders and use in other rough environments. The ability to test for multiple targets and internal control targets simultaneously, should allow for increased throughput and more consistent and controllable results.

It will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

In addition, where features or aspects of the invention are described in terms of Markush groups or other grouping of alternatives, those skilled in the art will recognize that the invention is also thereby described in terms of any individual member or subgroup of members of the Markush group or other group.

Also, unless indicated to the contrary, where various numerical values are provided for embodiments, additional embodiments are described by taking any 2 different values as the endpoints of a range. Such ranges are also within the scope of the described invention.

All references, patents, and/or applications cited in the specification are incorporated by reference in their entireties, including any tables and figures, to the same extent as if each reference had been incorporated by reference in its entirety individually.

TABLE 1
Exemplary Primers

| Oligo | | Conc. in | | Software |
|---|---|---|---|---|
| Name | Sequence 5′->3′ | PCR (nM) | Target | Design |
| AS005 | CAAACAGCCCAGTTACAATTACATTAG | 200 | pagA gene | Primer Express |
| AS006 | FAM-TXAATCCAGGAATCCTGCTCCATC | 200 | pagA gene | Primer Express |
| AS007 | CAGATAATGCATCGCTTGCTTTAG | 200 | capB gene | Primer Express |
| AS008 | HEX-TXGGATGAGCATTCAACATACCACG | 200 | capB gene | Primer Express |
| MM698 | HEX-XGATAATGCATCGCTTGCTTTAG | 150 | capB gene | Visual OMP |
| MM699 | GCTGTTTCCTCATCAATCCC | 150 | capB gene | Visual OMP |
| PN 1143 | FAM-TXCATGTCGGGGGCATATAAC | 100 | cya gene | Primer 3 |
| PN 1144 | TGCACCTGACCATAGAACG | 100 | cya gene | Primer 3 |
| PN 1323 | FAM-XCCTGCTCCATCTGATAATACTCTA | 100 | pagA gene | Visual OMP |
| PN 1324 | AGCAGGCAAGGACAGTG | 100 | pagA gene | Visual OMP |
| PN 1139 | Cy5-TXGCCTGCTGTGCTGTGT | 100 | IPC | Primer 3 |
| PN 1140 | TCGTGCGGTGCGTC | 100 | IPC | Primer 3 |
| PN 1141 | HEX-TXGCGCCGTAAAGAAGGTC | 150 | capB gene | Primer 3 |
| PN 1142 | CTACCCTGCGTTGCTCA | 150 | capB gene | Primer 3 |

FAM, 6-carboxyfluorescein
HEX, hexachlorofluorescein
Cy5, cyanine 5
X, 5-methyl-isocytosine

TABLE 2
Exemplary Panel of Organisms for Testing Analytical Specificity and Sensitivity

| | | | Strain |
|---|---|---|---|
| Organism | Strain Number | Organism | Number |
| Bacillus anthracis | 57 | Acinetobacter baumanni | 19606 |
| Bacillus anthracis | 108 | Alcaligenes xylosoxydans | 27061 |
| Bacillus anthracis | 183 | Bacteroides distasonis | 8503 |
| Bacillus anthracis | 205 | Bordetella bronchiseptica | 10580 |
| Bacillus anthracis | 4229 | Brucella abortus | |
| Bacillus anthracis | 4728 | Brucella melitensis | |
| Bacillus anthracis | Ames | Brucella canis | |
| Bacillus anthracis | BGC 8246/FTD 1064 | Brucella maris | |
| Bacillus anthracis | Buffalo | Brucella suis | |
| Bacillus anthracis | CDC 471 | Brucella neotomae | |
| Bacillus anthracis | CDC 607 | Brucella ovis | |
| Bacillus anthracis | Delta NH-1 | Budvicia aquatica | 35567 |
| Bacillus anthracis | Delta Sterne | Burkholderia cepacia | 25416 |
| Bacillus anthracis | English Vollum | Burkholderia pseudomallei | |
| Bacillus anthracis | FLA-V770 | Clostridium perfringens | 13124 |
| Bacillus anthracis | G-28 | Clostridium sporogenes | 3584 |
| Bacillus anthracis | Ger. LVS | Clostridium botulinum type B | |
| Bacillus anthracis | M | Clostridium botulinum type B | |
| Bacillus anthracis | N-99 | Clostridium botulinum type F | |
| Bacillus anthracis | N994 | Clostridium botulinum type C | |
| Bacillus anthracis | New Hampshire | Clostridium botulinum type D | |
| Bacillus anthracis | New Hampshire | Clostridium botulinum type E | |
| Bacillus anthracis | SK-102 | Comanonas acidovorans | 15668 |
| Bacillus anthracis | SK-128 | Enterococcus faecalis | 700802D |
| Bacillus anthracis | SK-162 | Enterobacter aerogenes | |
| Bacillus anthracis | SK-31 | Enterococcus durans | 6056 |
| Bacillus anthracis | SK-465 | Enterococcus faecalis | 29212 |
| Bacillus anthracis | SPS 97.13.079 | Escherichia coli | 25922 |
| Bacillus anthracis | SPS 97.13.213 | Francisella tularensis | |
| Bacillus anthracis | ST-1 | Francisella tularensis | |
| Bacillus anthracis | ST-15 | Francisella tularensis | |
| Bacillus anthracis | Sterne | Francisella tularensis | |
| Bacillus anthracis | V770 | Francisella tularensis | |
| Bacillus anthracis | V770-2P | Francisella tularensis | |
| Bacillus anthracis | V770-NP-1R | Haemophilus influenzae | 10211 |
| Bacillus anthracis | Vollum | Haemophilus influenzae | 51907D |
| Bacillus anthracis | Vollum 1B | Klebsiella pneumoniae | 13883 |
| Bacillus anthracis | Vollum-1 | Klebsiella oxytoca | 49131 |
| Bacillus cereus | 10876 | Klebsiella pneumoniae subsp. pneumonioae | 700721D |
| Bacillus cereus | 13061 | Listeria monocytogenes | 15313 |
| Bacillus cereus | 7039 | Moraxella cattaharalis | 25240 |
| Bacillus cereus | 12480 | Neisseria lactamica | 23970 |
| Bacillus cereus | 13472 | Pseudomonas aeruginosa | 17933D |
| Bacillus cereus | 13824 | Proteus mirabilis | 7002 |
| Bacillus cereus | 14603 | Proteus vulgaris | 49132 |
| Bacillus cereus | 14893 | Providencia stuartii | 33672 |
| Bacillus cereus | 15816 | Ralstonia pickettii | 27511 |
| Bacillus cereus | 19625 | Staphylococcus aureus | 35556D |
| Bacillus cereus | 19637 | Salmonella choleraesuis subsp. choleraesuis | 9150D |
| | | serotype Paratyphi | |
| Bacillus cereus | 21182 | Streptococcus pyogenes | 12344D |
| Bacillus cereus | 21366 | Serratia marcescens | 13880 |
| Bacillus cereus | 21634 | Shigella flexneri | 12022 |
| Bacillus cereus | 21769 | Shigella sonnei | 9290 |
| Bacillus cereus | 21768 | Staphylococcus aureus | 25923 |
| Bacillus cereus | 21771 | Staphylococcus aureus | 29213 |
| Bacillus cereus | 21772 | Staphylococcus hominis | 27844 |
| Bacillus cereus | 21770 | Staphylococcus aureus | 29247 |
| Bacillus cereus | 21928 | Stenotrophomonas maltophilia | 13637 |
| Bacillus cereus | 25621 | Streptococcus pyogenes | 19615 |
| Bacillus cereus | 27348 | Streptococcus pneumoniae | 33400 |
| Bacillus cereus | 27522 | Yersinia kristensenii | 33639 |
| Bacillus cereus | 27877 | Yersinia frederiksenii | 33641 |
| Bacillus cereus | 31293 | Yersinia kristensenii | 33638 |
| Bacillus cereus | 31429 | Yersinia pseudotuberculosis | 6904 |
| Bacillus cereus | 31430 | Yersinia ruckeri | 29908 |
| Bacillus cereus | 33018 | Yersinia pestis (Antigua; Pgm+) | |
| Bacillus cereus | 33019 | Yersinia pestis (Nairobi) | |
| Bacillus cereus | 43881 | Yersinia pestis (PBM19:Pgm+) | |
| Bacillus cereus | 53522 | Yersinia pestis (Pestoides B) | |
| Bacillus cereus | 55055 | Yersinia pestis (Pestoides F) | |
| Bacillus cereus | 700282 | Yersinia pestis Java 9 | |
| Bacillus cereus | 9139 | Yersinia pestis (CO92; PW) | |
| Bacillus coagulans | 7050 | | |
| Bacillus macerans | 8244 | | |
| Bacillus popilliae | 14706 | | |
| Bacillus subtilis | var. Niger | | |
| Bacillus thuringiensis | 35646 | | |
| Bacillus thuringiensis | 39152 | | |
| Bacillus thuringiensis | 10792 | | |
| Bacillus thuringiensis | 13366 | | |
| Bacillus thuringiensis | 13367 | | |
| Bacillus thuringiensis | 19266 | | |
| Bacillus thuringiensis | 19267 | | |
| Bacillus thuringiensis | 19268 | | |
| Bacillus thuringiensis | 19269 | | |
| Bacillus thuringiensis | 19270 | | |
| Bacillus thuringiensis | 29730 | | |
| Bacillus thuringiensis | 33679 | | |

TABLE 3
Positive control testing results from the pagA:capB:IPC and
cya:capB:IPC MultiCode-RTx Bacillus anthracis assays.

| | pagA:capB:IPC | | | | cya:capB:IPC | | | |
|---|---|---|---|---|---|---|---|---|
| | pagA - F1 - FAM | | capB - F2 - HEX | | cya - F1 - FAM | | capB - F2 - HEX | |
| | Cₜ | Tₘ | Cₜ | Tₘ | Cₜ | Tₘ | Cₜ | Tₘ |
| Mean | 37.4 | 79.2 | 38.3 | 81.8 | 31.8 | 76.2 | 35.4 | 80.1 |
| SD | 1.7 | 0.2 | 1.9 | 0.1 | 0.6 | 0.3 | 1.1 | 0.4 |
| % CV | 4.5% | 0.2% | 5.0% | 0.1% | 1.9% | 0.4% | 3.0% | 0.5% |

Eight reactions of each assay were run using 1 pg B. anthracis Ames DNA. The mean cycle threshold (Ct), melting temperature (Tm), standard deviation (SD) and percent coefficient of variation (% CV) are tabulated.

TABLE 4
Exemplary Primers with SEQ ID NOs

| Target | Sequence (5′->3′) | SEQ ID NO |
|---|---|---|
| pagA gene | FAM-TXAATCCAGGAATCCTGCTCCATC | SEQ. ID 1 |
| pagA gene | CAAACAGCCCAGTTACAATTACATTAG | SEQ. ID 2 |
| capB gene | HEX-TXGGATGAGCATTCAACATACCACG | SEQ. ID 4 |
| capB gene | CAGATAATGCATCGCTTGCTTTAG | SEQ. ID 3 |
| capB gene | HEX-XGATAATGCATCGCTTGCTTTAG | SEQ. ID 5 |
| capB gene | GCTGTTTCCTCATCAATCCC | SEQ. ID 6 |
| capB gene | HEX-TXGCGCCGTAAAGAAGGTC | SEQ. ID 7 |
| capB gene | CTACCCTGCGTTGCTCA | SEQ. ID 8 |
| cya gene | FAM-TXCATGTCGGGGGCATATAAC | SEQ. ID 9 |
| cya gene | TGCACCTGACCATAGAACG | SEQ. ID 10 |
| pagA gene | FAM-XCCTGCTCCATCTGATAATACTCTA | SEQ. ID 11 |
| pagA gene | AGCAGGCAAGGACAGTG | SEQ. ID 12 |
| Internal Control | Cy5-TXGCCTGCTGTGCTGTGT | SEQ. ID 13 |
| Internal Control | TCGTGCGGTGCGTC | SEQ. ID 14 |
| Internal Control | RNATCGTGCGGTGCGTCACACAGCACAGCAGGC | SEQ. ID 15 |

FAM, 6-carboxy-fluorescein
HEX, hexachlorofluorescein
Cy5, Cyanine 5
X, deoxy 5-methyl isocytidine

TABLE 5
Exemplary Formulations

| | | | | For 16 Samples |
|---|---|---|---|---|
| | | | | including 10% |
| Component | Concentration | Final Conc. | Per Rxn. (μl) | overage (μl) |
| 2X Solution Buffer | 2x | 1x | 12.5 | 220 |
| 50X Anthrax Primer | 50x | 1x | 0.5 | 8.8 |
| Mix | | | | |
| Titanium Taq | 50x | 1x | 0.5 | 8.8 |
| Internal Control DNA | N/A | N/A | 0.5 | 8.8 |
| Nuclease Free Water | N/A | N/A | 5.0 | 88 |
| | | Total Reaction | 20 | 352 |
| | | Mix Volume | | |
| | | Total Reaction | 25 (w/ 5 μl | 25 (20 μl rxn. mix per |
| | | Volume | Target) | tube + 5 μl Target) |

What is claimed is:

1. A method of detecting virulent bacteria in a sample, wherein the virulent bacteria include pX01 and pX02 nucleic acid, the method comprising:
a) amplifying the pX01 and pX02 nucleic acid, if present in the sample, with first and second primer pairs to provide amplification products, wherein at least one primer of the first primer pair specifically hybridizes to pX01 nucleic acid, and at least one primer of the second primer pair specifically hybridizes to pX02 nucleic acid, and at least one primer of each primer pair comprises a first non-natural base and a first label;
b) incorporating a second non-natural base into the amplification products, wherein the second non-natural base base-pairs with the first non-natural base and the second non-natural base is coupled to a second label;
c) observing a signal during amplification thereby detecting and quantifying the pX01 and pX02 nucleic acid in the sample.

2. The method of claim 1, wherein the virulent bacteria is a member of the Bacillus genus.

3. The method of claim 1, wherein the virulent bacteria is a strain of Bacillus anthracis.

4. The method of claim 1, wherein at least one primer of the first primer pair specifically hybridizes to a nucleic acid sequence selected from the group consisting of cya nucleic acid sequence, lef nucleic acid sequence, pagA nucleic acid sequence, atxA nucleic acid sequence, and pagR nucleic acid sequence.

5. The method of claim 4, wherein at least one primer of the first primer pair specifically hybridizes to a nucleic acid sequence selected from the group consisting of cya nucleic acid sequence and pagA nucleic acid sequence.

6. The method of claim 1, wherein at least one primer from the second primer pair specifically hybridizes to a nucleic acid sequence selected from the group consisting of capB nucleic acid sequence, cap C, nucleic acid sequence, capA nucleic acid sequence, dep nucleic acid sequence, and acpA nucleic acid sequence.

7. The method of claim 6, wherein at least one primer from the second primer pair specifically hybridizes to a capB nucleic acid sequence.

8. The method of claim 1, wherein the first non-natural base is iso-C or iso-G.

9. The method of claim 8, wherein the second non-natural base is the other of iso-C or iso-G.

10. The method of claim 1, wherein the first label comprises a fluorophore and the second label comprises a quencher.

11. The method of claim 10, wherein the at least one primer of each primer pair comprises a different fluorophore.

12. The method of claim 1, further comprising:
(d) amplifying an internal control nucleic acid to provide a control amplification product,
(e) detecting the internal control nucleic acid.

13. A method of detecting a virulent bacteria in a sample, wherein the virulent bacteria include pX01 and pX02 nucleic acid, the method comprising:
a) reacting a mixture that comprises:
(i) the sample;
(ii) a first oligonucleotide primer comprising a sequence complementary to the pX01 nucleic acid, a first non-natural base, and a first label;
(iii) a second oligonucleotide primer comprising a sequence complementary to the pX02 nucleic acid, a second non-natural base, and a second label; and
(iv) a nucleotide comprising a third non-natural base and a quencher, wherein the third non-natural base base-pairs with the first and second non-natural bases;
b) amplifying the pX01 and pX02 nucleic acid, if present in the sample, to generate labeled amplification products; and
c) observing a signal from the first label, the second label, or both labels during amplification thereby detecting the virulent bacteria in the sample.

14. The method of claim 13, wherein the virulent bacteria is a strain of Bacillus anthracis.

15. The method of claim 13, wherein the first oligonucleotide primer specifically hybridizes to a nucleic acid sequence selected from the group consisting of cya nucleic acid sequence, lef nucleic acid sequence, pagA nucleic acid sequence, atxA nucleic acid sequence, and pagR nucleic acid sequence.

16. The method of claim 13, wherein the second primer specifically hybridizes to a nucleic acid sequence selected from the group consisting of capB nucleic acid sequence, cap C, nucleic acid sequence, capA nucleic acid sequence, dep nucleic acid sequence and acpA nucleic acid sequence.

17. The method of claim 13, wherein the first oligonucleotide primer specifically hybridizes to a nucleic acid sequence selected from the group consisting of cya nucleic acid sequence and pagA nucleic acid sequence, and wherein the second oligonucleotide primer specifically hybridizes to a capB nucleic acid sequence.

18. The method of claim 13, wherein the first non-natural base and the second non-natural base are iso-C or iso-G, and the third non-natural base is the other of iso-C or iso-G.

19. The method of claim 13, wherein the first label comprises a fluorophore and the second label comprises a different fluorophore.

20. A kit comprising:
a) a first oligonucleotide primer comprising a sequence complementary to the pX01 nucleic acid, a first non-natural base, and a first fluorophore;
b) a second oligonucleotide primer comprising a sequence complementary to the pX02 nucleic acid, a second non-natural base, and a second fluorophore; and
c) a nucleotide comprising a third non-natural base and a quencher for the first and second fluorophores, wherein the third non-natural base base-pairs with the first and second non-natural bases.
